(19) United States
(12) Patent Application Publication
(10) Pub. No.: US 2017/0267755 A1
(43) Pub. Date: Sep. 21, 2017

(54) ISOLATED ANTI-MESOTHELIN ANTIBODIES, CONJUGATES AND USES THEREOF

(71) Applicant: SRI International, Menlo Park, CA (US)

(72) Inventor: Nathalie Scholler, Menlo Park, CA (US)

(21) Appl. No.: 15/461,309

(22) Filed: Mar. 16, 2017

Related U.S. Application Data
(60) Provisional application No. 62/308,883, filed on Mar. 16, 2016.

(51) Int. Cl. C07K 16/28; A61K 49/16; G01N 33/574; A61K 49/10

(52) U.S. Cl. CPC C07K 16/28; A61K 49/10; A61K 49/16; G01N 33/57492; C07K 2317/565; C07K 2317/569; C07K 2317/22; C07K 2317/92; G01N 2333/705

(57) ABSTRACT

Embodiments in accordance with the present disclosure include antibody binding domains that specifically bind to mesothelin. A nanobody or conjugate construct thereof can comprise the antibody binding domain comprising complementary determining region (CDR)1 of SEQ ID NO:05 or SEQ ID NO:08, CDR2 of SEQ ID NO:06 or SEQ ID NO:09, and/or CDR3 of SEQ ID NO:07 or SEQ ID NO:10. The nanobody or conjugate constructs of the nanobody can be used for diagnosis or treatment of diseases or conditions associated with overexpression of mesothelin.

### INCORPORATION-BY-REFERENCE OF MATERIAL SUBMITTED ELECTRONICALLY

Incorporated by reference in its entirety is a computer-readable nucleotide/amino acid sequence listing submitted concurrently herewith, and identified as follows: One 3,814 Byte ASCII (Text) file named “SRII104PA_Sequence_ST25” and created on Mar. 13, 2017.

### OVERVIEW

Antibodies are proteins that can be used by the immune system to detect, neutralize, and/or kill various target cells, such as tumor cells and pathogens, which may be harmful to the host organism. The antibody can recognize and bind to a unique molecule of the target cell, called an antigen, via a binding region of the antibody. An antibody bound to the antigen can directly or indirectly (e.g., by triggering other parts of the immune system), detect, neutralize, and/or kill the target cell. For example, the binding may block a part of a microbe that is essential for the target cell to invade and survive. In other examples, the binding may impede biological processes causing the disease or may activate macrophages to destroy the target cell.

Specifically, antibodies and antibody fragments are widely used in oncology for nanotechnology based diagnostic, therapeutic, and prognostic assays. As a particular example, the diagnosis and therapy of ovarian cancer (OC), the fourth leading cause of cancer deaths among women in the United States, can benefit from the development of immunosensors and targeted nanoparticles. Several cancer immunotherapies are being developed to target mesothelin, a differentially expressed cancer biomarker with limited normal expression that is upregulated in a variety of epithelial tumors. The cell surface-associated form of mesothelin is expressed at great levels in adenocarcinomas of the ovary and pancreas and in epithelial mesotheliomas compared to normal tissues, while mesothelin serum levels are elevated at diagnosis in most late stage OC patients and in most patients with malignant mesotheliomas (MM). Serum levels of mesothelin correlate with tumor size and increase during tumor progression, and the presence of mesothelin in MM pleural fluid can help to better discriminate mesothelioma from pleural metastasis. Due to a lack of accurate tools to diagnose early-stage disease, when a cure is still possible, the OC fatality-to-case ratio remains exceedingly high.

An antibody is generally a Y-shaped protein found in blood of humans and other vertebrates, and which belong to the immunoglobulin G (IgG) superfamily. There are five subclasses of antibodies, which include IgG, IgA, IgM, IgE, and IgD. Typically antibodies are made of various structural blocks and have two pairs of heavy chains and light chains. Each pair of a heavy chain and a light chain form a structure (e.g., like a lock) that fits a particle structure on an antigen, e.g., forms a binding region. While the general structures of different antibodies are similar, the binding region of the antibody is variable between the different antibodies and each of these variants can bind to different antigens. The heavy chains have one variable domain (V_{H}) followed by a constant domain C_{H}1, a hinge region and two more constant domains (C_{H}2 and C_{H}3). The light chains have one variable domain V_{L} and one constant domain C_{L}.

Various research has been devoted to recombinant antibodies. Monoclonal antibodies (mAbs) are produced using hybridoma-based techniques. By contrast, recombinant antibodies are monoclonal antibodies that are generated in vitro using synthetic genes. Recombinant antibodies that recognize mesothelin are advantageous for developing next generation diagnostic or therapeutic platforms and immunosensors because of the flexibility to incorporate various tags or functional groups for site-specific and oriented attachment of antibody fragments to surfaces.

Other types of antibodies are isolated from various animals and can be structurally different than human antibodies, such as camelids. Camelids include both conventional antibodies and a unique class of antibodies that lack a light chain and are composed of only heavy chains, sometimes referred to as camelid heavy-chain only antibodies (HcAbs). The binding activity of these HcAbs can be generated by a single variable domain named VHH, as opposed to traditional antibodies, where the paratope is assembled through the association of two variable domains (variable heavy (VH) and variable light (VL)).

### SUMMARY OF THE INVENTION

The present invention is directed to overcoming the above-mentioned challenges and others related to the methods and compositions that specifically bind to mesothelin, and can be used for diagnosis and therapy. Specific aspects are directed to isolated antibodies and conjugate constructs of the isolated antibodies, particularly single chain antibodies or nanobodies comprising a subject mesothelin binding domain. The isolated antibodies and conjugate constructs of the isolated antibodies can be used for diagnosis and treatment of diseases associated with mesothelin overexpression. The present invention is exemplified in a number of implementations and applications, some of which are summarized below as examples.

Antibodies in accordance with the present disclosure are based on camelid variable domains that specifically bind to mesothelin with an affinity of 15 or 30 nanomolar (nM). The antibodies can include isolated nanobodies (Nbs), such as single variable domain of camelid heavy-chain only antibodies (HcAbs).

In specific aspects, the inherent stability of Nbs which bind to mesothelin can be assessed, such as for multiple biomedical applications. Anti-mesothelin Nbs can be selected by phage display for specific binding to recombinant mesothelin conjugated to magnetic beads and screened by enzyme-linked immunosorbent assays (ELISA) for binding to plastic-immobilized mesothelin. The binding characteristics of candidate Nbs are characterized by flow cytometry using mesothelin-positive HeLa cells. The highest affinity Nb (Nb A1) can be modified by incorporating a C-terminal cysteine (Cys-Nb) to allow for bioconjugations using thiol-maleimide chemistry. In addition, Nb A1 can be transferred into a yeast-secreting system to produce site-specific biotinylated nanobodies (Bio-Nb). Both systems of conjugation (cysteine and streptavidin/biotin) can be used to characterize and validate the anti-mesothelin Nb and to generate functionalized nanoparticles. For example, a nanobody can be biotinylated by a process of covalently attaching a biotin to the Nb. Biotin (e.g., a linker) can bind to streptavidin and avidin. Binding of biotin to streptavidin and avidin results in stabilization i.e., resistance, to heat, pH, and proteolysis and can increase sensitivity of detection of protein, and can act as a label.

Various embodiments of the present disclosure are directed to an isolated nanobody (also referenced herein as “Nb” or “Nbs”) that specifically bind to human mesothelin with a particular affinity. The nanobodies are based on the single variable domain (e.g., the VHH domain) of camelid HcAbs (camelid heavy-chain only antibodies) specific for mesothelin. The Nbs and conjugate constructs of the Nb specifically bind to mesothelin and can be used for a variety of applications such as the detection and/or targeting of mesothelin for screening, diagnosis and/or treatment of a mesothelin-associated disease, disorders or conditions (e.g., cancer), or symptoms. The isolated nanobodies can have inherent in vivo and in vitro stability.

In specific embodiments, the isolated nanobody includes an antibody antigen binding domain which specifically binds to human mesothelin, and comprises complementarity determining region (CDR)1, CDR2 and CDR3, combinations as follows: SEQ ID NO:05, SEQ ID NO:06, and SEQ ID NO:07 or SEQ ID NO:08, SEQ ID NO:09, and SEQ ID NO:10. In various specific embodiments, the isolated nanobody comprises CDR1 of SEQ ID NO:05 or SEQ ID NO:08, CDR2 of SEQ ID NO:06 or SEQ ID NO:09, and CDR3 of SEQ ID NO:07 or SEQ ID NO:010.

In various embodiments the antigen binding domain comprises a variable region (e.g., VHH or V) comprising a sequence that is SEQ ID NO:02 or SEQ ID NO:04, and/or is a single domain antibody, and/or is an immune-conjugate, such as a chimeric antibody, chimeric antigen receptor T-cell receptors, antibody-drug conjugates (e.g. chemotherapeutic drugs like DM4, immunotoxins like PE38, etc.) or label conjugate (e.g. beads, metals, fluorescent labels, etc.). For ease of reference, the antibody having the antigen binding domain comprising SEQ ID NO:02 is sometimes referred to as G3A or Nb A1 and the antibody having the antigen binding domain comprising SEQ ID NO:04 is sometimes herein referred to as F10 or Nb C6.

Specific embodiments are directed to an isolated antibody (e.g., nanobody) that specifically binds to mesothelin and for use in a method of diagnosing and/or treating an organism, such as a mouse or human. The antibody can bind to mesothelin for the use in detecting and/or treating ovarian cancer, lung cancer, breast cancer, pancreatic cancer, and mesothelioma, among other diseases associated with overexpression of mesothelin. The antibody can comprise a variable region as disclosed in the attached Sequence List. For example, the antibody can comprise a variable region comprising at least one of SEQ ID NO:05, SEQ ID NO:06, and SEQ ID NO:07. In other embodiments, the antibody can comprise a variable region comprising at least one of SEQ ID NO:08, SEQ ID NO:09, and

SEQ ID NO:10. In some specific embodiments, the antibody includes CDR1 comprising SEQ ID NO:05 or SEQ ID NO:08, CDR2 comprising SEQ. ID NO:06 or SEQ ID NO:09, and CDR3 comprising SEQ ID NO:07 or SEQ ID NO:10.

A number of embodiments are directed to novel polynucleotides such as complementary double stranded deoxyribonucleic acid (cDNAs) and expression vectors, encoding a subject anti-mesothelin antigen binding domain, and cells comprising such polynucleotides, and non-human animals comprising such cells. Nb A1 and Nb C6 cDNA sequences are provided in SEQ ID NO:01 and SEQ ID NO:03, respectively. The polynucleotides may be operably linked to a heterologous transcription regulating sequence for expression, and may be incorporated into such vectors, cells, etc.

Various other embodiments are directed to methods of using the subject domains to bind and/or detect mesothelin, such as by administering the domain to a person determined to have cancer, and detecting resultant specific binding.

A number of embodiments are directed to the use of subject polynucleotides for the manufacture of a medicament for detecting or treating mesothelin-associated cancer or inhibiting tumor progression in a subject. Various embodiments are directed to conjugate construct, comprising a nanobody as described herein and that is modified.

Embodiments in accordance with the present disclosure include all combinations of the recited particular embodiments. Further embodiments and the full scope of applicability of the invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. All publications, patents, and patent applications cited herein, including citations therein, are hereby incorporated by reference in their entirety for all purposes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various example embodiments may be more completely understood in consideration of the following detailed description in connection with the accompanying drawings, in which:

FIG. 1 illustrates an example process for selecting an anti-mesothelin nanobody, in accordance with various embodiments;

FIG. 2 illustrates an example of result of phage titrations, in accordance with various embodiments,

FIGS. 3A-3C illustrates an example nanobody specificity of anti-mesothelin nanobody, in accordance with various embodiments,

FIGS. 4A-4B illustrate example characterization of a mesothelin epitope recognized by an example nanobody, in accordance with various embodiments;

FIGS. 5A-5C illustrate example characterization of an affinity of nanobodies for mesothelin, in accordance with various embodiments;

FIGS. 6A-6F illustrates examples images of immunofluorescence detection of mesothelin using conjugate constructs, in accordance with various embodiments;

FIGS. 7A-7G illustrates example immunotargeting with nanobody-based nanoparticles, in accordance with various embodiments;

FIGS. 8A-8B illustrate examples of experimentally determined stability of nanobodies, in accordance with various embodiments;

FIGS. 9A-9B illustrates example of nanobodies binding at physiological temperature, in accordance with various embodiments;

FIG. 10 illustrates example images of a mouse treated with human mesothelin human ovarian cancer cell line and an antibody, in accordance with various embodiments;

FIGS. 11A-11D illustrates example images of a mouse treated with mouse mesothelin ovarian cancer cell line and an antibody, in accordance with various embodiments;

FIGS. 12A-12C illustrates example images of a mouse treated with mouse mesothelin ovarian cancer cell line and an antibody, in accordance with various embodiments;

FIG. 13 illustrates example images of a mouse having no tumor that is treated with an antibody, in accordance with various embodiments;

FIGS. 14A-14I illustrates example images of mice treated with mesothelin human lung cancer cell line and an antibody, in accordance with various embodiments;

FIGS. 15A-15D illustrate example of detecting mesothelin-expresser ovarian cancer cells in vitro using an antibody, in accordance with various embodiments;

FIGS. 16A-16E illustrate examples images of detection of human ovarian cancer cells using an antibody, in accordance with various embodiment; and

FIGS. 17A-17E illustrate examples images of detection of mouse ovarian cancer cells using an antibody, in accordance with various embodiment.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

Aspects of the present disclosure are believed to be applicable to a variety of different types of compositions and methods related to isolated antibodies that specifically bind to mesothelin and can be used for diagnosis and/or treatment of organisms. In certain implementations, the antibodies include an isolated camelid, and conjugate constructs (e.g., forms) of the isolated camelid, that have been shown to beneficial when used in the context of binding to and/or for detecting mesothelin-expressing cells. In other implementations, isolated and/or conjugate constructs of the antibodies can be used for treatment of the organism, such as neutralizing or killing of the mesothelin-expressing cancer cells. While the present invention is not necessarily limited to such applications, various aspects of the invention may be appreciated through a discussion of various examples using this context.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same reference numerals may be used in different diagrams to refer to the same elements or additional instances of the same element.

Various embodiments in accordance with the present disclosure include isolated antibodies that specifically bind to mesothelin. Mesothelin, a cancer biomarker overexpressed in tumors of epithelial origin, is a target for diagnostic and targeting applications. Antibodies in accordance with the present disclosure can surprisingly be nanobodies (Nbs) derived from the immunization of llamas with mesothelin. Nbs are single chain or single domain antibodies (sdAbs). Immunizing the llamas with mesothelin can result in enriching the normal antibody repertoire of the llama by in vivo affinity maturation process and prior to creating a Nb gene library that can yield Nbs that have high affinity to mesothelin. The resulting antibody (e.g., Nb) in accordance with the present disclosure is smaller in size than a mAb or other antibody having a heavy chain and a light chain due to the single domain, which can increase the penetration ability and in vivo stability.

When Nbs are produced on their own, these minimal antibody fragments (13 kDa) are endowed with numerous properties that make them useful as a minimal binding unit for developing diagnostic immunosensors and therapeutic immunotherapies through antibody engineering. For example, despite their small size, reduced paratope and monovalent binding, these antibody fragments i) have affinities typical for regular monoclonal antibodies, ii) can bind small molecules and haptens, iii) show high production yields, extreme refolding capabilities and physical stability, and iv) can recognize buried cavities at antigen surfaces not accessible to regular monoclonal antibodies using a long complementarity determining region 3 (CDR3) hypervariable loop. Libraries of Nbs generated from immunized animals represent a rich source of antigen-specific, easy-to-produce, and stable antibody fragments that can be efficiently panned by phage display methods and easily fused to various tags allowing strong and oriented immobilization to various surfaces, including nanoparticles, for biomedical applications.

The functionalization and versatility of the nanobodies and conjugate constructs can be characterized using different site-specific functionalization approaches, including site-specific biotinylation or incorporating a free cysteine residue, for bioconjugation to superparamagnetic iron oxide nanoparticles and quantum dots using the biotin/streptavidin interaction or thiol-maleimide chemistry. The resulting antibodies exhibit versatility in targeting mesothelin, in particular as conjugate constructs described above, as the ability to recognize mesothelin in conventional immunophenotyping assays (e.g., flow cytometry, immunofluorescence, and western blot) and after bioconjugation is not hindered, providing reagents that can be used for diagnostic, therapeutic, and prognostic applications, such as for the detection and prognosis of ovarian cancer. In specific embodiment the biotinylated or cysteine-containing nanobody can be incorporated into nanosensors that recognize mesothelin. The sensitivity of immunosensors depends on the amount and functionality of the immobilized antibody or antibody fragment. Since site-specific immobilization produces nanoparticles or surfaces with a higher density of antigen binding sites in a productive orientation for antigen recognition the higher density of functional antibody fragments possible via site-directed coupling compared with natural IgG enhances the nanosensor response and decreases the detection limit. For more general and specific information related to site-specific immobilization produced nanoparticles, reference is made to Loch CM, et al, “Use of high density antibody arrays to validate and discover cancer serum biomarkers”, Mol Oncol, 1(3):313-20 (2007), and to Sukhanova, et al., “Oriented conjugates of single-domain antibodies and quantum dots: toward a new generation of ultrasmall diagnostic nanoprobes”, Nanomedicine 8(4):516-25 (2012).

In specific embodiments, a phage-display library of Nbs that are amplified from B-cells of a llama immunized with human recombinant mesothelin is generated. Anti-mesothelin Nbs are initially selected for specific binding to recombinant mesothelin in the liquid phase by magnetic sorting, screened by enzyme-linked immunosorbent assay (ELISA) assays, and validated by flow cytometry using mesothelin-expressing cell lines. Two nanobodies (Nb A1 (sometimes referred to as “G3A”) and Nb C6 (sometimes referred to as “F10”)) can be selected on the basis of affinity (K_{D}=15 and 30 nM, respectively for the Nb A1 and C6). The Nbs, in some specific embodiments, are further modified by adding either a cysteine to permit maleimide-based bioconjugations, or a sequence for the site-specific metabolic addition of a biotin in vivo. Both systems of conjugation (thiol-maleimide and streptavidin/biotin) are used to characterize and validate Nbs and to functionalize nanoparticles. The anti-mesothelin Nbs can be used to detect native and denatured mesothelin in various diagnostic applications, including flow cytometry, western blot, immunofluorescence, and optical imaging. Anti-mesothelin Nbs are novel, cost effective, small, and single domain reagents with high affinity and specificity for the tumor associated antigen mesothelin that can be simply bioengineered for multiple bioconjugation strategies for attachment to nanoparticles or modified surfaces. These anti-mesothelin Nbs are useful in conventional diagnostic assays and for nanoparticle-based molecular targeting and novel next generation immunosensor applications.

Unless the context indicates otherwise, the term “antibody” is used in the broadest sense and specifically covers antibodies (including full length monoclonal antibodies) and antibody fragments so long as they specifically bind mesothelin. An antibody molecule is usually monospecific, but may also be described as idiospecific, heterospecific, or polyspecific. Antibody molecules bind by means of specific binding sites to specific antigenic determinants or epitopes on antigens. The subject antibodies may be single chain or single domain antibodies (sdAbs) or nanobodies (Nbs). “Antibody fragments” comprise a portion of a full length antibody, generally the antigen binding or variable region thereof. Examples of antibody fragments include Fab, Fab′, F(ab′).sub.2, and Fv fragments; diabodies; linear antibodies; single chain antibody molecules; and multispecific antibodies formed from antibody fragments.

Natural and engineered antibody structures are well known in the art. For general and specific information regarding engineering antibody structures to form conjugate constructs, reference is made to Strohl et al., “Therapeutic antibody engineering: Current and future advances driving the strongest growth area in the pharmaceutical industry”, Woodhead Publishing Series in Biomedicine No. 11, October 2012; Holliger et al. Nature Biotechnol 23, 1126-1136 (2005); and Chames et al. Br J Pharmacol. 2009 May; 157(2): 220-233, each of which are hereby incorporated for their teachings.

Monoclonal antibodies (mAbs) may be obtained by methods known to those skilled in the art. The mAbs of the invention may be of any immunoglobulin class including IgG, IgM, IgE, IgA, and any subclass thereof. A hybridoma producing a mAb may be cultivated in vitro or in vivo. High titers of mAbs can be obtained in in vivo production where cells from the individual hybridomas are injected intraperitoneally into mice, such as pristine-primed BALB/c mice to produce ascites fluid containing high concentrations of the desired mAbs. MAbs of isotype IgM or IgG may be purified from such ascites fluids, or from culture supernatants, using column chromatography methods well known to those of skill in the art.

An “isolated polynucleotide” refers to a polynucleotide segment or fragment which has been separated from sequences which flank it in a naturally occurring state, e.g., a deoxyribonucleic acid (DNA) fragment which has been removed from the sequences which are normally adjacent to the fragment, e.g., the sequences adjacent to the fragment in a genome in which it naturally occurs. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by polymerase chain reaction (PCR) or restriction enzyme digestion) independent of other sequences. It also includes a recombinant DNA, which is part of a hybrid gene encoding additional polypeptide sequence.

A “construct” means any recombinant polynucleotide molecule such as a plasmid, cosmid, virus, autonomously replicating polynucleotide molecule, phage, or linear or circular single-stranded or double-stranded DNA or ribonucleic acid (RNA) polynucleotide molecule, derived from any source, capable of genomic integration or autonomous replication, comprising a polynucleotide molecule where one or more polynucleotide molecule has been linked in a functionally operative manner, i.e. operably linked. A recombinant construct will typically comprise the polynucleotides as disclosed in embodiments herein that are operably linked to transcriptional initiation regulatory sequences and that can direct the transcription of the polynucleotide in the intended host cell. Both heterologous and non-heterologous (i.e., endogenous) promoters can be employed to direct expression of the nucleic acids of the invention.

A “vector” refers to any recombinant polynucleotide construct that may be used for the purpose of transformation, i.e. the introduction of heterologous DNA into a host cell. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

An “expression vector” as used herein refers to a nucleic acid molecule capable of replication and expressing a gene of interest when transformed, transfected or transduced into a host cell. The expression vectors comprise one or more phenotypic selectable markers and an origin of replication to ensure maintenance of the vector and to, if desired, provide amplification within the host. The expression vector further comprises a promoter to drive the expression of the polypeptide within the cells. Suitable expression vectors may be plasmids derived, for example, from pBR322 or various pUC plasmids, which are commercially available. Other expression vectors may be derived from bacteriophage, phagemid, or cosmid expression vectors.

Various specific embodiments are directed to isolated antibodies (e.g., nanobodies) and conjugate constructs of the antibodies that can bind to mesothelin with a K_{D} of about 30 nM or 15 nM. In specific embodiments, the nanobodies can be used to detect and/or screen for diseases or conditions associated with overexpression of mesothelin. Further, the nanobodies and/or conjugate construct can be used in treatment and/or prevention of such diseases, such as by neutralizing or killing the target mesothelin-expressing cells. The nanobodies disclosed herein can also be isolated and/or made into conjugate constructs, such as humanized nanobodies or antibodies, derived from reference nanobodies according to standard methods known in the art. For example, the conjugate constructs of the nanobodies can include engineered derivatives of nanobodies as disclosed herein. Engineered derivatives of the nanobodies can be made using techniques employed on a regular basis in laboratories of one of ordinary skill in the art. These engineered derivatives include but are not limited to antibody-drug conjugates (ADCs); chimeric antigen receptors (CARs); bispecific antibodies that include but are not limited to bispecific T-cell engagers (BiTEs), TrioMabs, dual-affinity retargeting molecules (DARTs), tandem diabodies (TandAbs), and other; engineered human variable or constant antibody domains of the heavy or light chain (including single-chain antibody derivatives); immunotoxins that include but are not limited to cytolytic fusion proteins and cytotoxic proteins derived from microorganisms; and radioconjugates. Antibody genes can be engineered to enhance the affinity of the nanobodies and the conjugate construct for their mesothelin target via an affinity maturation process.

A number of embodiments are directed to the genetic modification or other modification of the nanobodies, such as by conjugation to another molecule or part thereof to form conjugates of the nanobodies. An example of such conjugation construct includes a Nb as disclosed herein (comprising SEQ ID NO:02, also referenced as “Nb A1” or “G3A” herein) that is modified to incorporate a C-terminal cysteine (Cys-Nb). Other example conjugate constructs include modifying a Nb to produce a site-specific biotinylated nanobody (Bio-Nb) (e.g., by transferring the Nb into a yeast-secreting system to produce the biobody). The conjugate constructs comprising cysteine (e.g., a C-terminal cysteine) or biotin (e.g., to permit further binding to streptavidin) can be used to characterize and validate the anti-mesothelin Nb and to generate Nb-functionalized nanoparticles.

Mesothelin refers to mesothelin proteins or polypeptides which remain intracellular as well as secreted and/or isolated extracellular mesothelin protein, e.g., soluble mesothelin and variants thereof. The nanobodies can be specific for mesothelin obtained from a species, e.g., human or mice. Mesothelin is a 40 kDa cell-surface glycosylphosphatidylinositol (GPI)-linked glycoprotein. The human mesothelin protein is synthesized as a 69 kD precursor which is then proteolytically processed. The 30 kD amino terminus of mesothelin is secreted and is referred to as megakaryocyte potentiating factor. An epitope, as used herein, refers to an antigenic determinant. An epitope can be part of the antigen (e.g., mesothelin) that is recognized by the immune system and further specially binds to antibodies, B-cells and/or T-cells. An antigenic determinant is formed from chemical groups and/or peptide sequences on the antigen and is capable of eliciting an immune response. The antigenic determinant may be linear, such as comprising a consecutive sequence of residues within an amino acid sequence, although embodiments are not so limited.

Surprisingly, embodiments in accordance with the present disclosure include selecting and isolating anti-mesothelin nanobodies that specifically bind to mesothelin based on camelid VHH domain. Nanobodies (Nbs) can preserve the antigen selectivity of whole antibodies, are extremely stable, can be produced more economically, and straightforward antibody bioengineering techniques can be used to allow oriented nanoparticle conjugation as compared to whole antibodies. By contrast, conventional immunoglobulins G (IgG) with a molecular weight of one hundred fifty kDa may not be well-suited for nanoparticle targeting purposes, since they yield very large bioconjugates which often impedes their efficiency. Moreover, the conditions used for mAb bioconjugation often result in random mAb orientation on the nanoparticle surface.

As previously described, Nbs are isolated that specifically and efficiently bind mesothelin using phage display technology. Specifically, llamas are immunized with recombinant (human) mesothelin and a Nb library is constructed. The Nb library, which in specific embodiments can be around 10⁸ clones, is constructed using peripheral blood cells of llama immunized with recombinant mesothelin and respective Nbs are selected using the recombinant human mesothelin. Two rounds of direct selection using phage antibody produced with helper phage KM13 can be used to pan over epoxy-coated paramagnetic beads previously incubated with mesothelin. Enrichment in the number of phages that recognize mesothelin is detected between the first and second round of selection. For example, non-specific phage-Nbs are discarded and the mesothelin-specific phage are amplified for a second round of selection. After each round of selection, clones can be screened, such as via ELISA using recombination mesothelin.

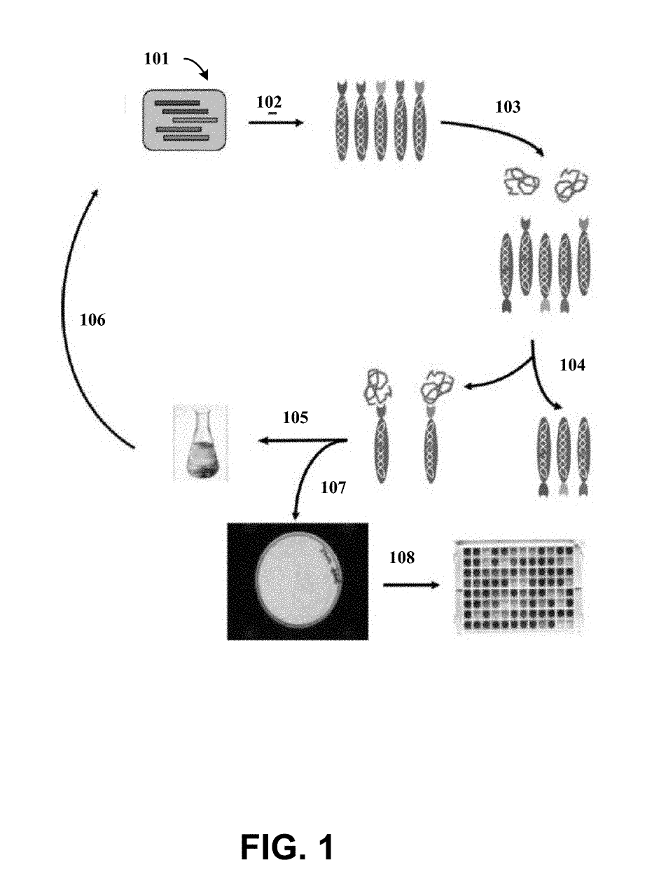

The relation and/or process of isolating Nbs can also be made readily apparent in an illustration, such as illustrated by FIG. 1. For example, FIG. 1 illustrates an example of the above-described process for selecting an anti-mesothelin nanobody, in accordance with various embodiments. As previously described, the Nb library is constructed and Nbs are selected from the library at 101 and 102. Two rounds of direct selection can be used (e.g., 105 and 106), and at each round, non-specific phage-Nbs are discarded at 103 and the mesothelin-specific phage are amplified (such as, for a second round of selection), at 104. After each round of selection, the clones are screened at 107 and 108. Accordingly, FIG. 1 shows an example process for isolating Nbs that specifically and efficiently bind to mesothelin using phage display technology, as previously described above.

In specific experimental embodiments, a phage-ELISA based screening procedure can be performed after the first round of affinity selection using biotinylated mesothelin immobilized on streptavidin plates can reveal that eighty-two out of ninety-three clones (88%) are positive. After the second round of selection, all clones picked from the output recognized mesothelin and produced background signals on control antigens. Forty-five out of ninety-three clones are assayed by flow cytometry for binding to mesothelin expressed on the plasma membrane of HeLa cells (mesothelin positive) or to Jurkat cells (mesothelin negative). Thirty-seven out of forty-five clones bound exclusively to HeLa cells. Sequence analyses of the twenty clones displaying the highest mean fluorescence intensities revealed two independent Nbs named A1 (representing 95% of binders) and C6 (5% of binders). The presence of an arginine on position forty-five can confirm the camelidae nature of these single domain antibodies. Nb A1 (sometimes referred to as G3A) comprises a variable region, including CDR1 comprising SEQ ID NO:05, variable region CDR2 comprising SEQ ID NO:06, and variable region CDR3 comprising SEQ ID NO:07. Nb C6 (sometimes referred to as F10) comprises a variable region, including CDR1 comprising SEQ ID NO:08, variable region CDR2 comprising SEQ ID NO:09, and variable region CDR3 comprising SEQ ID NO:10.

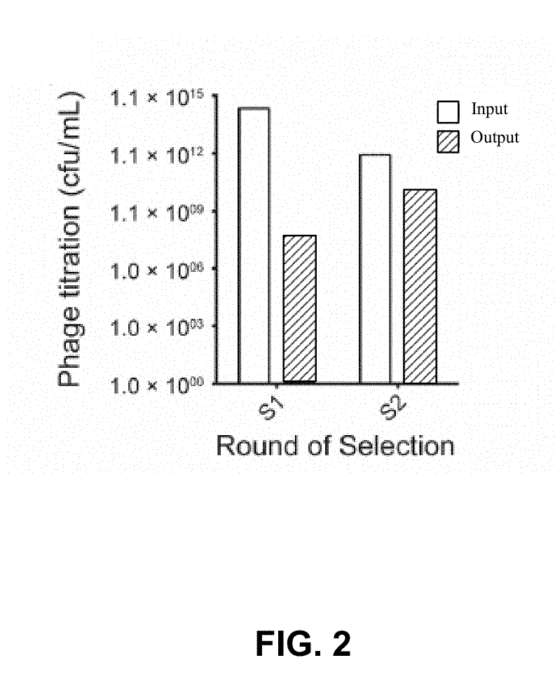

The relation of phage titrations and the resulting enrichment in the number of phages that can recognize mesothelin detected between the first and the second round of selection can also be made readily apparent in an illustration, such as illustrated by FIG. 2. Specifically, FIG. 2 illustrates example results of phage titrations after each round of selection as illustrated by FIG. 1. As previously described, enrichment in the number of phages that recognize mesothelin can be detected between the first and the second round of selection. Accordingly, FIG. 2 shows an example graph illustrating the detected number of phages that recognize mesothelin between the first and second round of selection and which are enriched, as previously described above.

In certain embodiments, the nanobodies or the conjugate construct of the nanobody (e.g., compound) disclosed herein can be modified. For example, the nanobody amino acid sequences SEQ ID NO:02; SEQ ID NO:04, or an amino acid sequence comprising one or more of (i) a CDR1 comprising the amino acid sequence of SEQ ID NO:05 or SEQ ID NO:08; (ii) a CDR2 comprising the amino acid sequence of SEQ ID NO0:6 or SEQ ID NO:09; and (ii) a CDR3 comprising the amino acid sequence of SEQ ID NO:07 or SEQ ID NO:10 can have one or more modifications. The resulting modified nanobodies or conjugate construct of the nanobody can specifically bind to mesothelin (e.g., retain the functional properties of the nanobodies). As is well known in the art, certain sequence modifications can be made that do not impact antigen binding. Modifications can be introduced into an amino acid sequence by standard techniques known in the art, such as site-directed mutagenesis and PCR-mediated mutagenesis. As further described herein, example modifications include amino acid substitutions, additions and deletion, such as replacing one or more amino acid residues within the CDR regions of a nanobody or a conjugate construct of this disclosure with other amino acid residues. The modified nanobody or conjugate construct can be tested for retained function.

The Nb specificity, in experiment embodiments, can be characterized by flow cytometry on cell lines with different mesothelin expression levels. Nbs containing a C-terminal hexahistidine tag can be produced in the periplasm of E. coli and purified by immobilized ion metal affinity chromatography. Final yields can be in the range of fifty mg/L culture for the two clones. Sodium dodecyl sulfate polyacrylamide gel electrophoresis (SDSPAGE) analysis demonstrates a degree of purity greater than a threshold (greater than 95%). Nanobodies can be assayed by flow cytometry for binding to ovarian cancer cells (OVCAR-3 and SK-OV-3), cervix adenocarcinoma cells (HeLa) or to prostate carcinoma cells (22Rv1). Mesothelin expression can be initially assessed on each cell line using anti-mesothelin mAb K1. The ovarian cancer SK-OV-3 show a low mesothelin expression while the prostate carcinoma cell line 22Rv1 does not express a detectable antigen level. Nb A1 (G3A) binding profiles are similar to mAb K1 despite its monovalency. Cell binding is also observed with Nb C6, which can be at a lesser extent than Nb A1, as no binding is observed on the mesothelin low SK-OV-3 cell line. No binding of mAb K1, Nb A1, and/or Nb C6 is detected on the prostate carcinoma cell line 22Rv1. Taken together, this confirms that both clones specifically bind to mesothelin.

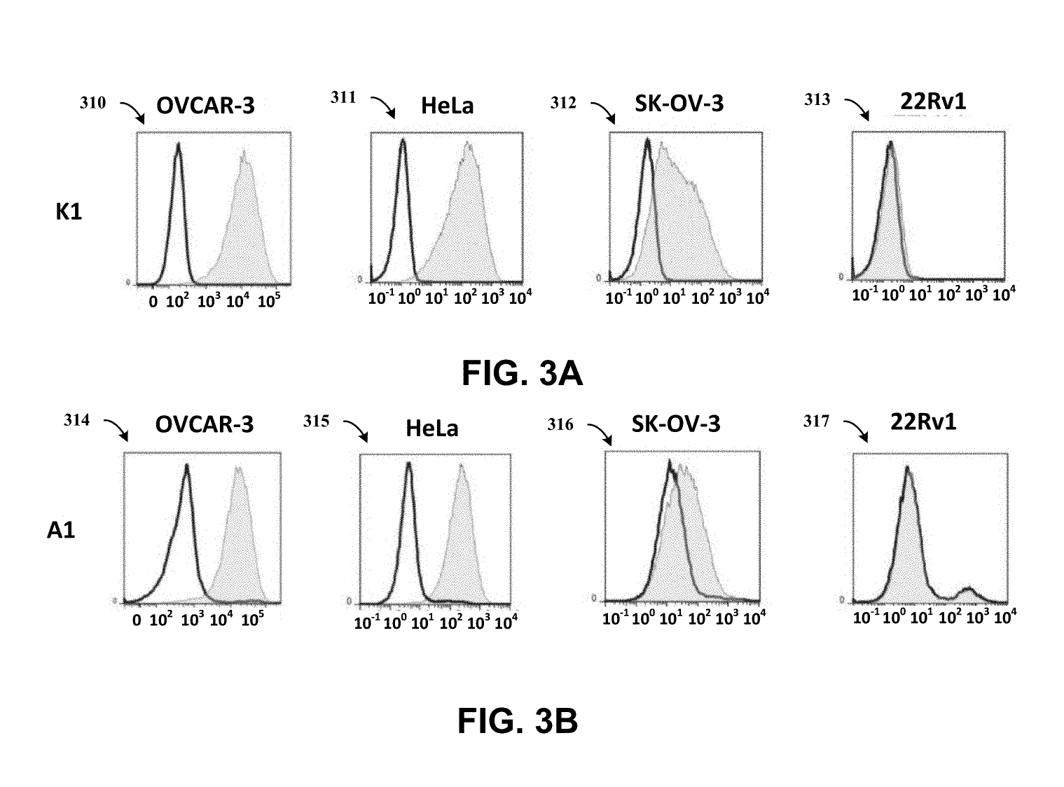

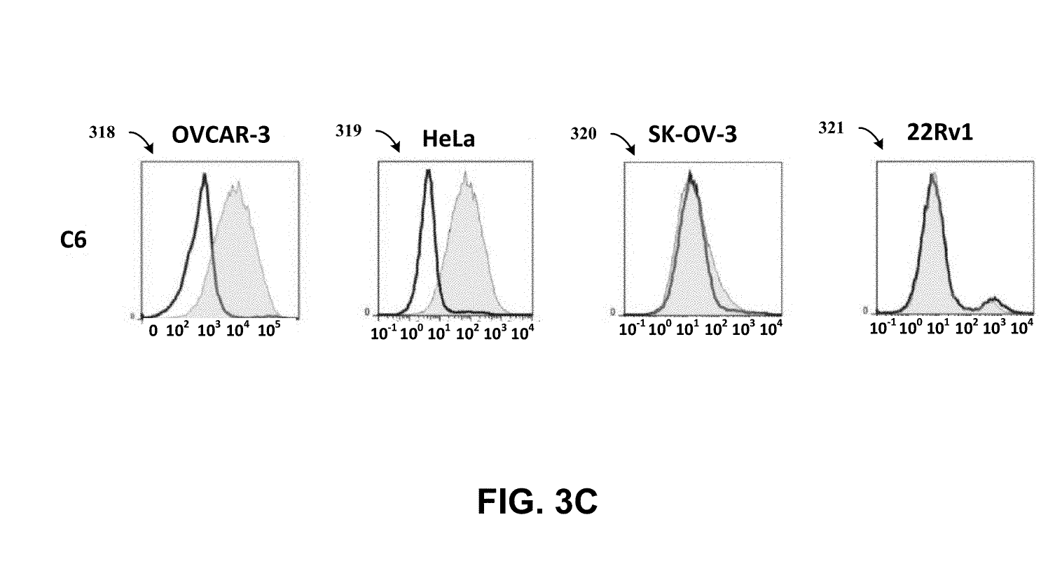

This relation of the specificity (e.g., binding) of Nbs in accordance with the present disclosure can also be made readily apparent in illustrations, such as the graphs illustrated by FIGS. 3A-3C. For example, FIGS. 3A-3C show example graphs illustrating specificity of an anti-mesothelin antibody and nanobodies, in accordance with various embodiments. The profiles illustrated in each of FIGS. 3A-3B illustrate the cell lines incubated with a secondary antibody as the solid black line and incubated with the respective antibody and/or nanobody followed by the secondary antibody as the filled histogram. FIG. 3A illustrates example binding profiles of mAb K1 to cell lines OVACR-3 (e.g., graph 310), HeLa (e.g., graph 311), SK-OV-3 (e.g., graph 312), and 22Rv1 (e.g., graph 313). The mAb K1 binding profiles suggest that OVCAR-3 and HeLa cells overexpress mesothelin, as illustrated by graphs 310 and 311 of FIG. 3A. Overexpressing mesothelin refers to or includes cells that express mesothelin at a greater rate than normal cells (e.g., non-diseased cells). The ovarian cancer SK-OV-3 cell line show a low mesothelin expression while the prostate carcinoma cell line 22Rv1 does not express a detectable antigen level, as illustrated by graphs 312 and 313 of FIG. 3A, as described above.

FIG. 3B illustrates example binding profiles of Nb A1 to cell lines OVACR-3 (e.g., graph 314), HeLa (e.g., graph 315), SK-OV-3 (e.g., graph 316), and 22Rv1 (e.g., graph 317). As described above, Nb A1 (sometimes referred to as G3A) binding profiles are similar to mAb K1 despite its monovalency.

FIG. 3C illustrates example binding of Nb C6 to cell lines OVACR-3 (e.g., graph 318), HeLa (e.g., graph 319), SK-OV-3 (e.g., graph 320), and 22Rv1 (e.g., graph 321). As described above, cell binding is also observed with Nb C6 (sometimes referred to as F10), but to a lesser extent than Nb A1 since no binding is observed on the mesothelin low SK-OV-3 cell line. No binding of mAb K1, Nb A1, or Nb C6 is detected on the prostate carcinoma cell line 22Rv1.

Accordingly, FIGS. 3A-3C show example results of flow cytometry on cell lines with different mesothelin expression levels, which confirms that both clones specifically bind to mesothelin, as previously described above.

In specific embodiments, the ability of the nanobodies Nb A1 and Nb C6 to cross-compete for binding to mesothelin can be characterized. Nanobodies and conjugate constructs thereof can cross compete for binding to mesothelin with any of the anti-mesothelin nanobodies disclosed herein. To determine if two Nbs share the same or part of their epitope, the phage nanobodies (phage-Nb) A1 and C6 and an irrelevant phage-Nb are assayed by flow cytometry for binding to HeLa cells in the presence of serial dilutions of purified Nb C6. As expected, phage-Nb C6 competes with Nb C6. A competitive binding is also observed between phage-Nb A1 and Nb C6, which indicates that the two clones bind the same or a proximal mesothelin epitope. The same result can be obtained by the reverse experiment, which assayed phage-Nb C6 binding to HeLa cells in the presence of serial dilutions of Nb A1, confirming the competition between the two clones. To further characterize the epitope, an immunoblot is performed using mammalian cell culture supernatant containing a recombinant human mesothelin (Msln-Ig) fusion protein. After reducing sodium dodecyl sulfate polyacrylamide gel electrophoresis (SDS-PAGE) and transfer to Polyvinylidene difluoride (PVDF) membrane, the recombinant Msln-Ig is detected using Nb A1 and the commercial monoclonal antibody K1. Detection of the recombinant protein by anti-human IgG (H+L) antibody can be used as a positive control.

The three antibodies detect the same band, which indicates that K1 and the Nbs (e.g., Nb A1 and Nb C6) recognize a linear epitope. These results demonstrate that isolated nanobodies, such as Nb A1, can be used for immunoblotting procedures.

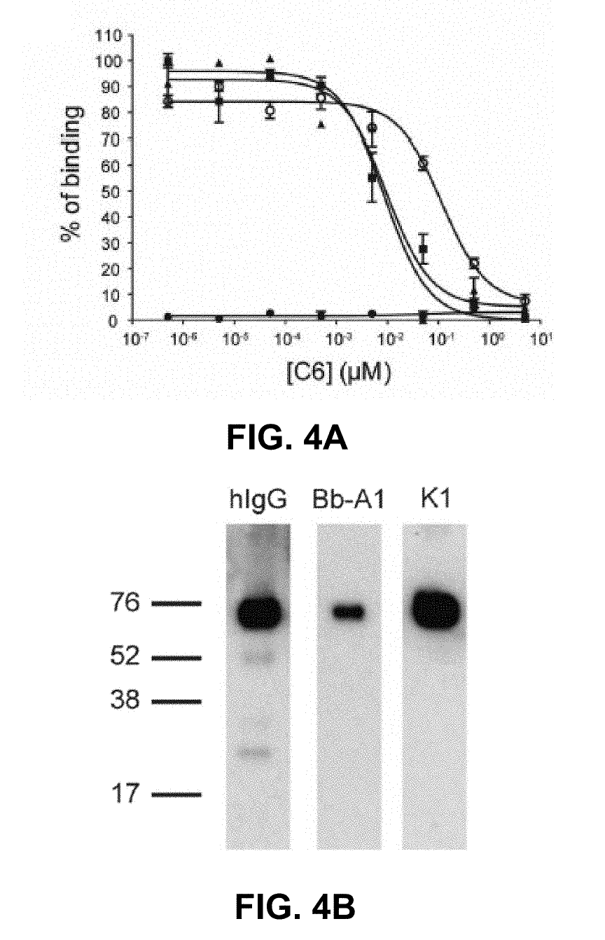

This relation of cross competing of Nbs in accordance with the present disclosure can also be made readily apparent in illustrations, such as the graphs illustrated by FIGS. 4A-4B. FIGS. 4A-4B illustrate example characterization of a mesothelin epitope recognized by an example nanobody, in accordance with various embodiments. Various competition assay known in the art or as described herein can be used to identify a nanobody or conjugate construct that competes with another one or more of the nanobodies or conjugate constructs described herein that specifically bind to mesothelin. In certain embodiments, such a competing nanobody or conjugate construct binds to the same epitope (e.g., a linear or a conformational epitope) that is bound by a nanobody or conjugate construct described herein. Specific example methods for a competition assay is provided in the experimental/more detailed embodiments section herein.

FIG. 4A illustrates example results of characterizing the cross-reactivity of nanobodies epitopes. In a specific embodiment, to determine if the two Nbs share the same or part of their epitope, the phage-Nb A1 (e.g., represented by ▴) and phage-Nb C6 (e.g., represented by ▪) and an irrelevant phage-Nb (e.g., represented by ) are assayed by flow cytometry for binding to HeLa cells in the presence of serial dilutions of purified Nb C6. As expected, phage-Nb C6 competes with Nb C6. A competitive binding is also observed between phage-Nb A1 and Nb C6, which indicates that the two clones bind the same or a proximal mesothelin epitope. The same result can be obtained by the reverse experiment, which assayed phage-Nb C6 binding to HeLa cells in the presence of serial dilutions of Nb A1, confirming the competition between the two clones. Commercial antibody mAb K1 (e.g., represented by ∘) can be added at constant and non-saturating concentration and detecting with a secondary antibody (e.g., PE-goat anti-mouse IgG antibody) by flow cytometry. Error bars represent the standard deviation of experiments performed in triplicate.

FIG. 4B illustrates an example result of mammalian cell culture supernatants immunoblotted against human IgG, Bb A1 and a commercial mAb K1. To further characterize the epitope, an immunoblot can be performed using mammalian cell culture supernatant containing a recombinant human mesothelin (Msln-Ig) fusion protein. After reducing SDS-PAGE and transfer to PVDF membrane, the recombinant Msln-Ig is detected using Nb A1 and the commercial monoclonal antibody K1. Detection of the recombinant protein by anti-human IgG (H+L) antibody can be used as positive control. The three antibodies detected the same band, which indicates that both mAb K1 and the Nbs (e.g., Nb A1 and Nb C6) recognize a linear epitope. These results demonstrate that Nb A1 can be used for immunoblotting procedures.

Accordingly, FIGS. 4A-4B show example results of flow cytometry used to determine if two Nbs share the same or part of their epitope and that indicates that mAb K1 and the Nbs recognize the linear epitope of mesothelin, as previously described above.

The Nbs in accordance with the present disclosure demonstrate a binding specificity for binding to mesothelin (e.g., affinity). For example, the nanobody or conjugate thereof that specifically binds to mesothelin or specifically binds to mesothelin with a particular affinity (e.g., a high affinity) can refer to a nanobody or conjugate thereof that binds to mesothelin with a K_{D} of about 30 nM or less or about 15 nM or less. In specific embodiments, the Nb A1 and Nb C6 have a K_{D} of approximately 15 nM and 30 nM, respectively.

In specific experimental embodiments, the affinity of the two Nbs, Nb A1 and Nb C6, can determined by flow cytometry using HeLa cells. Binding to HeLa cells can be detected using flow cytometry after incubation with various concentrations of biotinylated Nbs followed by PE-labeled streptavidin. K_{D} values are determined by the equation K_{D}=a *Fₘₐₓ in which “a” is the regression line and Fₘₐₓ is the maximum of fluorescence. Despite of their monovalency, as described above, Nb A1 has an apparent K_{D} of approximately 15 nM while Nb C6 had an apparent K_{D} of 30 nM. These affinity constants are comparable to the affinity of the commercial anti-mesothelin bivalent IgG antibody K1. In addition, the higher affinity and maximum mean fluorescence intensity (MFI) achieved with Nb A1 is consistent with its predominant representation in the phage display output and the larger fluorescence shift seen with flow cytometry.

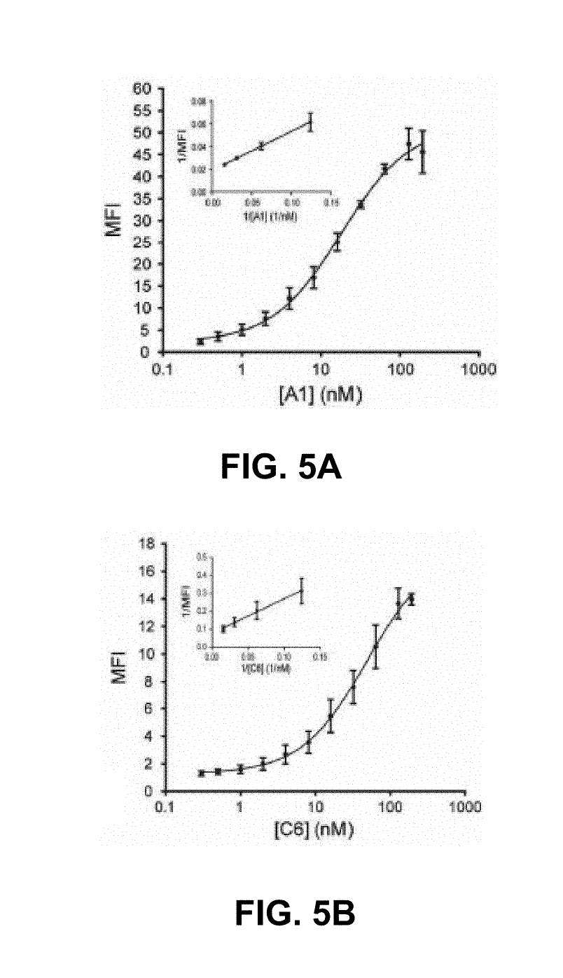

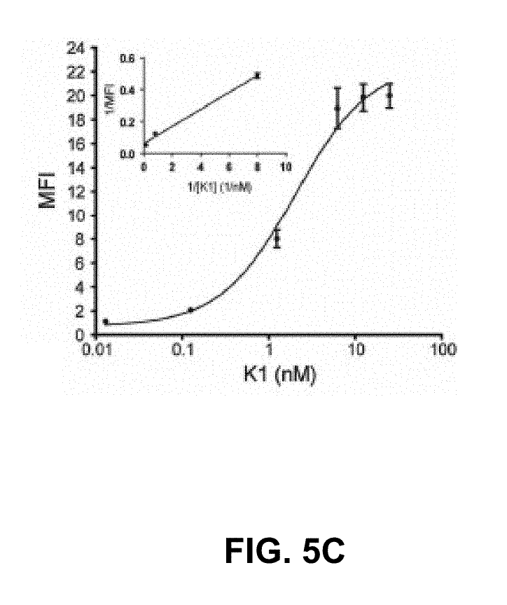

This relation of the affinity of Nbs on cells in accordance with the present disclosure can also be made readily apparent in illustrations, such as the graphs illustrated by FIGS. 5A-5C. FIGS. 5A-5C illustrate example graphs that characterize of an affinity of nanobodies and an antibody for mesothelin, in accordance with various embodiments. In specific embodiments, the affinity of the nanobodies A1 and C6 for binding to mesothelin on plasma membrane of cells can be determined. For example, FIG. 5A illustrates a graph that characterizes the affinity of Nb A1 for binding to mesothelin. FIG. 5B illustrates a graph that characterizes the affinity of Nb C6 for binding to mesothelin. And, FIG. 5C illustrates a graph that characterizes the affinity of mAb K1 for binding to mesothelin. Accordingly, FIGS. 5A-5C show example results of determining the affinity of the two Nbs by flow cytometry and using HeLa cells, as previously described above.

Clinical application of personalized medicine in cancer therapy using novel molecularly targeted platforms requires reliable tumor phenotyping. Immunofluorescence assays on frozen or formalin fixed, paraffin embedded sections from a multicellular tumor spheroid can be used to test the reactivity of biobody (Bb) A1, a metabolically and site-specific biotinylated version of Nb A1. Biobody A1 specifically and efficiently binds recognizes mesothelin in frozen sections compared to a control section incubated with only the secondary antibody. In contrast, Bb A1 shows poor reactivity on fixed, paraffin embedded sections prior to antigen retrieval, which can be enhanced by antigen retrieval at high pH. These results show that enzymatically biotinylated nanobodies can bind mesothelin by immunofluorescence on frozen and paraffin-fixed sections, thus providing a flexible approach to phenotyping tumors for novel immunotargeting-based in vivo diagnostics and therapeutics.

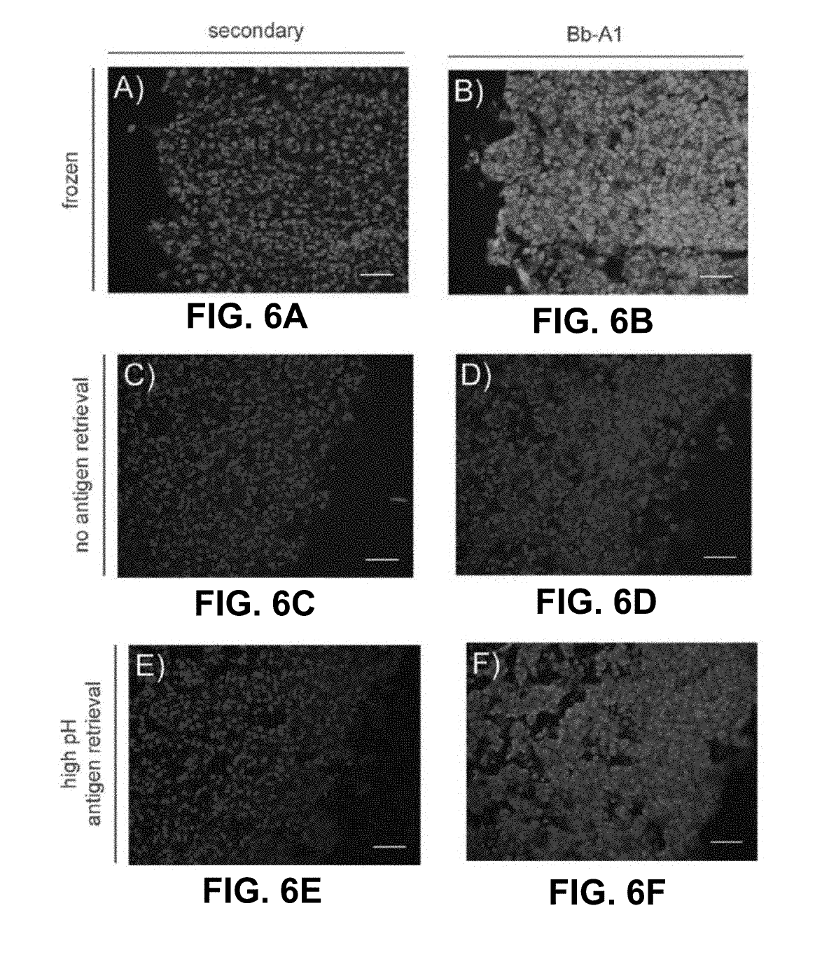

This relation of immunofluorescence detection of mesothelin using Nbs in accordance with the present disclosure can also be made readily apparent in illustrations, such as the images illustrated by FIGS. 6A-6F. FIGS. 6A-6F illustrate example images showing immunofluorescence detection of mesothelin using a conjugate construct of nanobodies, in accordance with various embodiments. An engineered conjugate construct of a nanobody disclosed herein can be generated. In some embodiments, the conjugate construct can be generated by performing biotinylation of the antibody to form a biobody. In specific embodiments, the conjugate construct includes a metabolically and site-specifically biotinylated version of Nb A1, e.g., Bb A1 as described above. Clinical application of personalized medicine in cancer therapy using novel molecularly targeted platforms requires reliable tumor phenotyping. Immunofluorescence assays on frozen or formalin fixed, paraffin embedded sections from a multicellular tumor spheroid can be used to test the reactivity of biobody (Bb) A1. Biobody A1 specifically and efficiently binds to (e.g., recognizes) mesothelin in frozen sections (e.g., FIG. 6B) compared to a control section incubated with only the secondary antibody (e.g., FIG. 6A). In contrast, Bb A1 showed poor reactivity on fixed, paraffin embedded sections prior to antigen retrieval (e.g. FIG. 6D), which can be enhanced by antigen retrieval at high pH as illustrated by FIG. 6F. These results show that enzymatically biotinylated nanobodies can bind mesothelin by immunofluorescence on frozen and paraffin-fixed sections, thus providing a flexible approach to phenotyping tumors for novel immunotargeting-based in vivo diagnostics and therapeutics.

More specifically, FIGS. 6A and 6B illustrate immunofluorescence detection from human cells (e.g., human ovarian cancer spheroids prepared using A1847 cells) and frozen in optimal cutting temperature compound (OCT). FIG. 6A illustrates the human (A1847) cells treated with a secondary antibody alone and FIG. 6B illustrates the human (A1847) cells treated with the Bb A1 and secondary antibody. FIGS. 6C-F illustrate immunofluorescence detection, such as from human ovarian cancer spheroids prepared using A1847 cells as fixed and paraffin embedded. FIGS. 6C and 6E illustrate the A1847 cells treated with a secondary antibody alone and FIGS. 6D and 6F illustrate the A1847 cells treated with the Bb A1 and secondary antibody. As illustrated by FIGS. 6E and 6F, the antigen can be retrieved at a particular pH values (as compared to no antigen retrieval in FIGS. 6C and 6D). Accordingly, FIGS. 6A-6F show example results of testing reactivity of Bb A1 using the immunofluorescence assays on frozen or formalin fixed, paraffin embedded sections from a multicellular tumor spheroid, as previously described above.

The biotintylated Nbs can mediate targeting of superpamagnetic iron oxide nanoparticles to mesothelin. For example, Bb A1 can self-assembled out of the crude yeast culture media onto streptavidin labeled superparamagnetic iron oxide nanoparticles (SPION) for fluorescent detection. The human ovarian cancer cell line C30 can be used as a negative control for nonspecific binding evaluation. All nanoparticles showed negligible fluorescence over the background cellular autofluorescence, which indicates a low level of nonspecific binding. In contrast, clear fluorescence shifts can be observed with Bb A1-functionalized nanoparticles on the A1847 human ovarian cancer cell line that overexpresses mesothelin. As a negative control, untargeted nanoparticles showed fluorescence levels that corresponded to background autofluorescence.

To further demonstrate the versatility of Nbs as nanoparticle targeting reagent, Nb A1 is modified to include a C-terminal cysteine residue (Cys-A1) for site-specific attachment to nanoparticles through thiol-maleimide coupling. Adding a free cysteine did not impair the Nb binding to HeLa cells because flow cytometry showed a large fluorescence shift of approximately 1.5 log units. The binding activity of the resulting conjugated QD (e.g., one day) is further characterized on cells grown in chamber slides. Carboxyfluorescein succinimidyl ester (CFSE) can be used to fluorescently label cells on the slides. Optical imaging at ten and fifty nM Cys-A1/QD concentrations demonstrates differential binding of the Cys-A1/QD bioconjugates to mesothelin positive (A1847) compared to mesothelin negative (C30) cells.

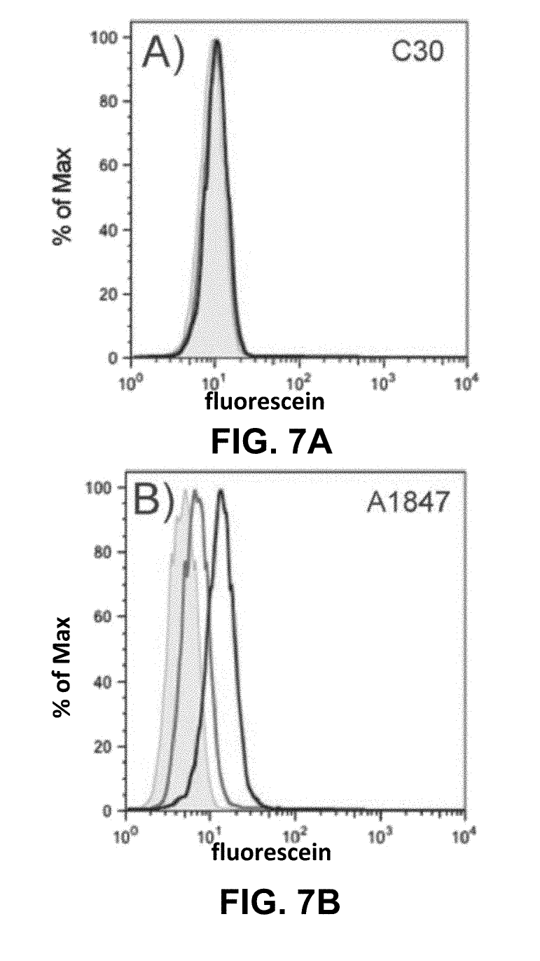

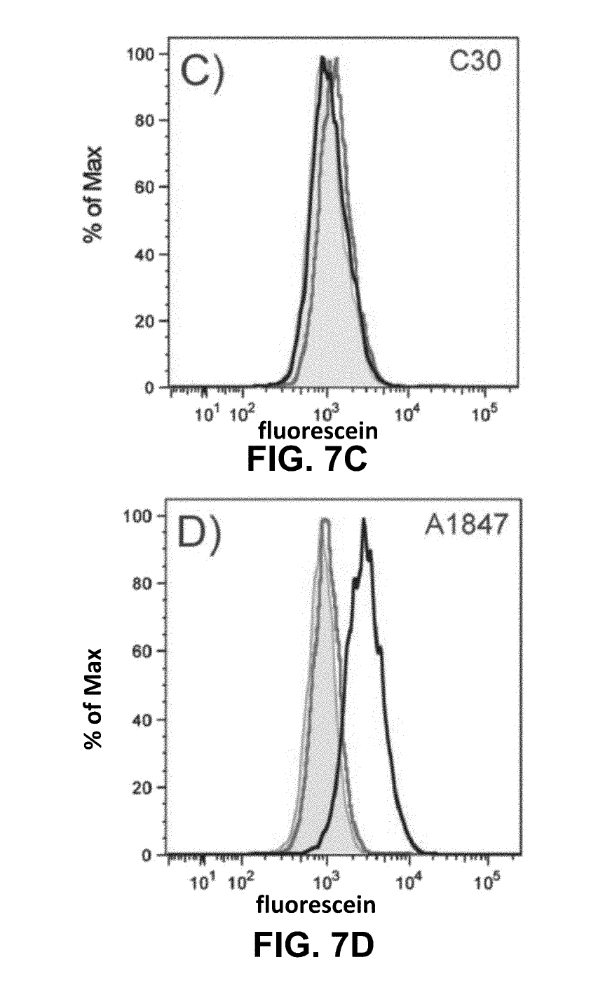

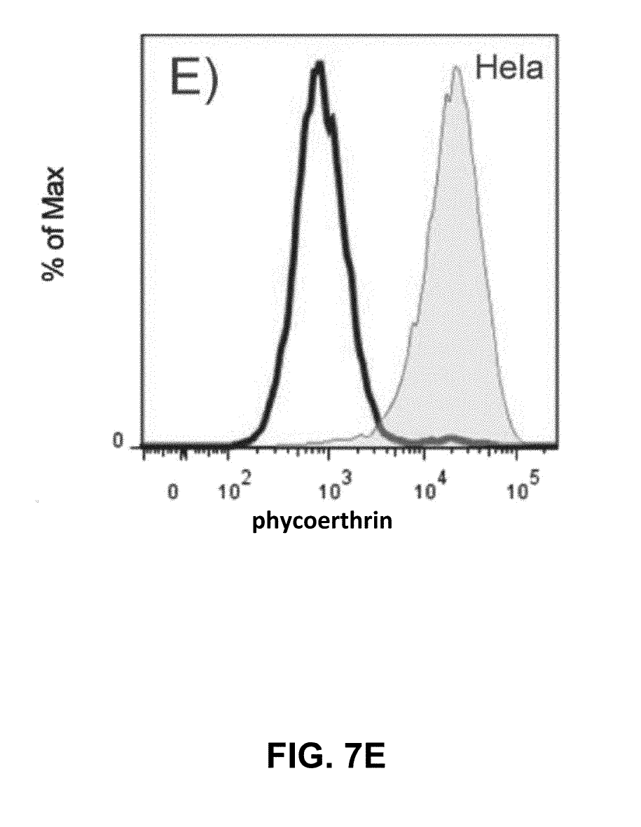

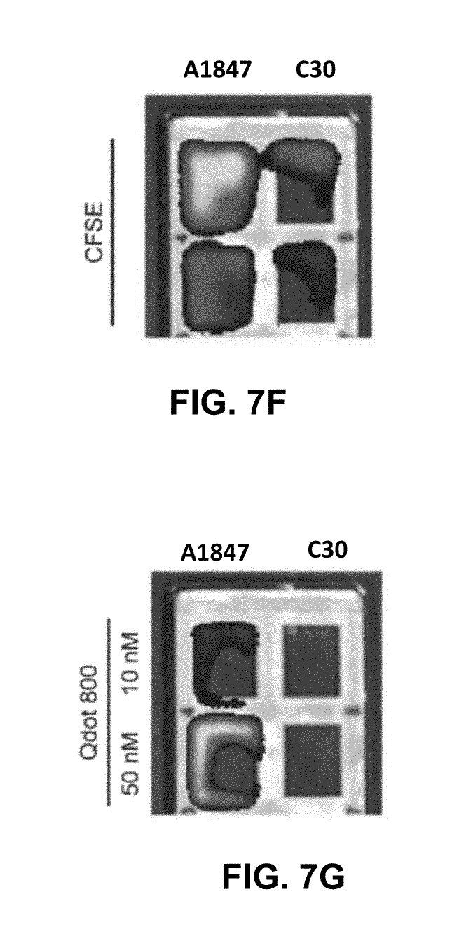

The relation of Nb mediated targeted of nanoparticles to mesothelin in accordance with the present disclosure can also be made readily apparent in illustrations, such as illustrated by FIGS. 7A-7G. FIGS. 7A-7G illustrate example immunotargeting with nanobody-based nanoparticles, in accordance with various embodiments, and as described above. FIGS. 7A and 7C illustrate human ovarian cancer cell line C30 being used as a negative control for nonspecific binding evaluation. FIGS. 7B and 7D illustrate fluorescent shifts observed by Bb A1-functionalized nanoparticles and K1 (black line) on the A1847 human ovarian cell line that overexpresses mesothelin. The lighter (grey) line illustrates the negative control which is untargeted nanoparticles and that corresponds to background fluorescence.

As further previously described, to further demonstrate the versatility of Nbs as nanoparticle targeting reagent, Nb A1 is modified to include a C-terminal cysteine residue (Cys-A1) for site-specific attachment to nanoparticles through thiol-maleimide coupling. Adding a free cysteine did not impair the Nb binding to HeLa cells because flow cytometry showed a large fluorescence shift of approximately 1.5 log units, as illustrated by FIG. 7E. The binding activity of the resulting conjugated QD (e.g., one day) is further characterized on cells grown in chamber slides. Carboxyfluorescein succinimidyl ester (CFSE) can be used to fluorescently label cells on the slides. Optical imaging at ten and fifty nM Cys-A1/QD concentrations demonstrates differential binding of the Cys-A1/QD bioconjugates to mesothelin positive (A1847) compared to mesothelin negative (C30) cells, as illustrated by FIGS. 7F and 7G. These results demonstrate that low concentrations of nanobodies can be used to detect the expression of mesothelin on living cells, without being hindered by non-specific binding to the cell surface.

Biotinylated Nbs mediate targeting of superparamagnetic iron oxide nanoparticles to mesothelin. Human ovarian cancer cell line C30 (e.g., illustrated by FIG. 7A and FIG. 7C) that lack mesothelin expression and cell line A1847 (e.g., illustrated in FIG. 7B and FIG. 7D) that express mesothelin can be incubated with a commercial mAb K1 antibody and analyzed by flow cytometry. The fluorescence intensity from an isotype control or untargeted SPION (gray line) is near the background fluorescence. Both mAb K1 and SPION immunotargeted to mesothelin using Bb A1 (black line) show a fluorescence increase compared to unstained cells. The gray filled histogram in FIGS. 7A-7D represents the background cellular autofluorescence of unstained cells. FIG. 7E illustrates the binding of Cys-A1 demonstrated using flow cytometry of HeLa cells after incubation with secondary antibody (black line) or His tagged Cys A1 followed by secondary antibody (gray filled histogram). Specificity of Cys A1 bioconjugated to quantum dots. Optical imaging can be used to detect CFSE-labeled mesothelin positive cells (A1847), as illustrated by FIG. 7F and mesothelin negative cells (C30) and mesothelin expression on cells using Cys A1 conjugated Qdot800, as illustrated by FIG. 7G.

Accordingly, FIGS. 7A-7G show example results of demonstrating the versatility of Nbs as nanoparticle targeting reagent using fluorescent detection of Bb A1-functionalized nanoparticles on A1847 human ovarian cell line and modifying Nb A1 to form Cys-A1, as previously described above.

### More Detailed/Experimental Embodiments

In these examples, llamas are immunized with mesothelin, an important cancer biomarker, to enrich the normal antibody repertoire by in vivo affinity maturation prior to creating a Nb gene library that yielded Nbs with low nanomolar affinities. The versatility of mesothelin targeting nanobodies is demonstrated using two different site-specific functionalization approaches (e.g., site-specific biotinylation or incorporating a free cysteine residue) for bioconjugation to nanoparticles using the biotin/streptavidin interaction or thiolmaleimide chemistry to superparamagnetic iron oxide nanoparticles and quantum dots.

In addition to the diagnostic applications demonstrated here, such as for the early detection and prognosis of ovarian cancer, the biotinylated or cysteine-containing Nb A1 can be advantageously incorporated into nanosensors that recognize mesothelin. The sensitivity of immunosensors depends on the amount and functionality of the immobilized antibody or antibody fragment. Since site-specific immobilization produces nanoparticles or surfaces with a higher density of antigen binding sites in a productive orientation for antigen recognition, the higher density of functional antibody fragments possible via site-directed coupling compared with natural IgG enhances the nanosensor response and decreases the detection limit.

Llama Immunization and VHH Library Construction

A young adult male llama is immunized subcutaneously at days one, twenty, forty one and sixty two with sixty five μg of recombinant human, soluble mesothelin protein produced as previously described. The VHH library can be constructed as previously described.

Selection of Nbs by Phage Display

Phages from the VHH library are produced. For general and specific information on VHH libraries and production of phages, reference is made to Behar G, et al., “Isolation and characterization of anti-FcgammaRIII (CD16) llama single-domain antibodies that activate natural killer cells”, Protein Eng Des Sel, 2008, 21(1):1-10, which is herein fully incorporated by reference for its teaching. Mesothelin conjugated to epoxy-coated paramagnetic beads (Dynabeads M-450 Epoxy, Invitrogen) can be used for two sequential rounds of immunoselection to identify phages that specifically recognize mesothelin. To label the Dynabeads, an aliquot (one hundred μL) is washed with 0.1 M sodium phosphate buffer (NaPi) and resuspended in one hundred μL of NaPi. Recombinant mesothelin (ten μg) is added to the beads and the solution is gently rotated for forty-eight hours at four degrees Celsius (C). For more general and specific information on adding recombinant mesothelin to beads, reference is made to Bergan, et al., “Development and in vitro validation of anti-mesothelin biobodies that prevent CA125/Mesothelin-dependent cell attachment”, Cancer Lett 255(2):263-74 (2007). Beads are washed three times by magnetic isolation with one mL of phosphate-buffered saline (PBS)/0.1% Tween-20 and then three times with one mL of PBS before being incubated with one mL of PBS/2% milk for two hours at room temperature. Mesothelin conjugated beads are resuspended with the phage preparation pre-incubated in PBS/2% milk. The solution is gently rotated for two hours at room temperature before being washed nine times with PBS/0.1% Tween-20, nine times with one mL of PBS, and then incubated with five hundred μL of trypsin (one mg/mL) for thirty minutes at room temperature. Eluted phage-Nbs are resuspended in five hundred μL of PBS and incubated without shaking with five mL of log phase TG1 cells which are subsequently plated on 2YT/ampicillin (one hundred μg/mL)/2% glucose (2YTAG) in 243×243 dishes (Nalgene Nunc). Ninety three colonies from the first round of selection and one hundred ninety two colonies from the second round of selection are picked, grown overnight in 96-well plates containing 200 μL 2YTAG and stored at negative eighty degrees C. after the addition of 15% glycerol. The remaining colonies are harvested from the plates, suspended in five mL of 2YTAG and used to produce phages for the next round of selection.

ELISA Screening of Phage-Nbs

Infected TG1 cells (five μL) from masterplates are used to inoculate one hundred-fifty μL of 2YTAG in 96-well plates. Colonies are grown for two hours at 37 degrees C. under shaking (nine hundred rotations per minute (rpm)), then fifty μL of 2YT containing 2×10⁸ M13KO7 helper phage are added to each well and incubated for thirty minutes at thirty seven degrees C. without shaking. Plates are centrifuged for ten minutes at 1200xg and bacterial pellets are resuspended in one hundred fifty μL of 2YT containing ampicillin (one hundred μg/mL) and kanamycin (fifty μg/ml), 2YTAK. Colonies are grown for sixteen hours at thirty degrees C. under shaking (nine hundred rpm). Phage containing supernatants are tested for binding to recombinant mesothelin by ELISA. Fifty μg of mesothelin is biotinylated in vitro using the EZ-Link Micro NHS-PEO4-Biotinylation Kit (Pierce) according to the manufacturer's recommendations. Biotinylated recombinant mesothelin (1.4 μg/mL) is bound to streptavidin-coated 96-well microplates for sixteen hours with PBS/2% milk. Fifty microliters of phage supernatant is added to fifty μL PBS/2% milk and incubated for one hour at room temperature in the ELISA microplate. Bound phages are detected at A405 using a peroxidase-conjugated monoclonal anti-M13 mouse IgG.

Cell Culture

The human cervix adenocarcinoma HeLa cell line can be obtained from the American Type Culture Collection (ATCC) and are cultured in Dulbecco's modified Eagle's medium supplemented with 10% heat-inactivated fetal bovine serum (FBS). Jurkat cells from ATCC are cultured in RPMI-1640 with 10% FBS. The SK-OV-3 and OVCAR-3 human ovarian adenocarcinoma cell lines are obtained from ATCC and cultured in Dulbecco's modified eagle's medium (DMEM) with 10% FBS and RPMI-1640 with 20% FBS, respectively. The 22Rv1 human prostate carcinoma cell line can be cultured in RPMI-1640 with 10% FBS. Ovarian cancer cell lines (C30 and A1847) from the University of Pennsylvania Ovarian Cancer Research Center are cultured in RPMI-1640 media with 10% FBS containing 1% penicillin/streptomycin (one hundred Units/mL penicillin and one hundred μg/mL streptomycin). Human embryonic kidney 293 cells from ATCC, which are transfected to secrete a chimeric protein containing the extracellular portion of mesothelin and an IgG hinge (293-Msln-Ig), are cultured in DMEM media with 10% FBS containing fifty μg/mL hygromycin B and 1% penicillin/streptomycin. All cell lines were maintained at thirty seven degrees C. under a humidified 5% CO2 atmosphere.

Screening of Phage-Nbs on Mesothelin-Positive Cells by Flow Cytometry

Phage-containing supernatants can be tested for binding to HeLa cells (mesothelin positive) and Jurkat cells (mesothelin negative). Flow cytometry is performed after incubating 5×10⁵ cells with fifty μL of phage-containing supernatants for one hour at four degrees C. under shaking (nine hundred rpm). Phage binding is detected by incubation with a primary monoclonal anti-M13 mouse IgG (ten μg/mL, GE Healthcare Life Sciences) followed by a phycoerythrin (PE)-labeled F(ab)′2 goat anti-mouse IgG (H+L) secondary antibody (Santa Cruz Biotechnology). Analyses can be carried out using a MACSQuant® Analyzer (Miltenyi Biotec) with FlowJo software. Phages displaying mean fluorescence intensity (MFI) two times above the negative control can be considered as mesothelin-specific phages.

Nanobody Sequencing, Production and Purification

DNA sequences of mesothelin-specific phages are determined by GATC Biotech AG (Applied Biosystems). One Nb from each identified family is selected, produced in E. coli strain BL21DE3, and subsequently purified. Overnight cultures in 2YTAG are diluted into 2YT (fifty mL) supplemented with two mM MgSO4, 0.05% glucose, 0.5% glycerol, 0.2% lactose and one hundred μg/mL ampicillin to obtain an OD600 of 0.1. Bacteria are grown for two hours at 37° C. then for sixteen hours at 30° C. under shaking (nine hundred rpm). Cells are harvested by centrifugation at 3000×g for twenty minutes at four degrees C. and the pellet is kept overnight at negative twenty degrees C. The pellet is resuspended in five mL of room temperature Bug Buster Extraction Reagent (Novagen) supplemented with ten μL of lysozyme (10 mg/mL) and 0.5 μL of benzonase (two hundred fifty U/μL). After incubation for thirty minutes at room temperature with gentle shaking, Nbs are purified by TALON metal-affinity chromatography (Clontech) and concentrated by ultrafiltration with Amicon Ultra 5000 MWCO (Millipore). The protein concentration are determined spectrophotometrically using the Bio-Rad DC protein assay (Bio-Rad Laboratories).

Cell Binding Experiments by Flow Cytometry

Nb and the anti-mesothelin monoclonal antibody K1 (Santa Cruz Biotechnology) are used to perform cell binding experiments by flow cytometry. Immunofluorescence assays are performed by incubating 5×10⁵ indicator cells (SK-OV-3, OVCAR-3, or 22Rv1) with Nb A1 (0.5 μg/mL), Nb C6 (0.5 μg/mL) or mAb K1 (0.4 μg/mL) for one hour at four degrees C. with shaking (nine hundred rotations per minute). Nb binding to each cell line is detected by incubation with a mouse F(ab)′2 anti-6His antibody (one μg/mL) followed by phycoerythrin-goat anti mouse IgG antibody (PE-GAM). An irrelevant Nb is used as a negative control. Binding of mAb K1 is detected by incubation with PE-GAM. PE-GAM is directly used as a negative control.

Immunofluorescence Competition Assay

Competition assays between nanobodies Nb A1 (e.g., SEQ ID NO:02) and Nb C6 (e.g., SEQ ID NO:04) can be performed by incubating 5×10⁵ HeLa cells with various concentrations of Nb A1 (from 0.5 μM to 5 μM) and a 1/200 dilution of the phage-Nb C6. The same experiment can be performed with various concentrations of Nb C6 (from 0.5 μM to five μM) and a 1/500 dilution of the phage-Nb A1. The binding of phage-Nbs is detected by incubation with monoclonal anti-M13 mouse IgG (ten μg/mL) followed by incubation with PE-GAM.

Affinity Measurements of Nb

Briefly, fifty μg of each Nb are biotinylated chemically using the EZ-Link Micro NHSPEO4-Biotinylation Kit. After incubation of mesothelin-positive HeLa cells (5×10⁵) with various concentrations of biotinylated Nbs for one hour at four degrees C. under shaking (nine hundred rpm), Nbs binding is detected by flow cytometry following incubation with (PE)-labeled streptavidin. The K_{D} values are determined by the equation:

1/(F−F_{back})=1/Fₘₐₓ+(K_{D}/Fₘₐₓ)(1/[antibody]),

in which F represents the fluorescence unit, F_{back}=background fluorescence and Fₘₐₓ is estimated from the data. The slope of the regression line is (a)=K_{D}/Fₘₐₓ so K_{D}=a*Fₘₐₓ.

Cloning and Expression of Soluble, Site-Specific Biotinylated Nb A1.

Site-specifically biotinylated nanobody A1 (named Bb A1) can be biosynthetically produced following an established protocol developed for scFv. For more general and specific information related to biosynthetic production, reference is made to Scholler, et. al, “Method for generation of in vivo biotinylated recombinant antibodies by yeast mating”, J Immunol Methods 317(1-2):132-43, 2006; and Zhao A, et. al, “Rapid isolation of high-affinity human antibodies against the tumor vascular marker Endosialin/TEM1, using a paired yeast-display/secretory scFv library platform”, J Immunol Methods 363(2):221-32, 2011, both of which are herein fully incorporated for their teachings. Briefly, the Nb A1 sequence can be PCR amplified to incorporate terminal sequences for homologous recombination with the p416-BCCP vector containing a biotin ligase recognition sequence. Linearized p416-BCCP vector and PCR product are chemically transformed into haploid Saccharomyces cerevisiae cells (YVH10) which are subsequently mated with haploid yeast containing a plasmid coding for the Escherichia coli biotin ligase for antibody secretion into the yeast culture supernatant after galactose induction. The site-specifically biotinylated molecules are named biobodies (Bb), as previously discussed.

Sodium Dodecyl Sulfate Polyacrylamide Gel Electrophoresis (SDS-PAGE) and Western Blotting of 293-Msln-Ig Culture Supernatant

To obtain a chimeric protein containing the extracellular portion of mesothelin and an IgG hinge, 293-Msln-Ig cells are grown to confluency, washed with Dulbecco's phosphate-buffered saline (DPBS), incubated in DMEM lacking FBS until the cells started to detach, and the culture supernatant is clarified by centrifugation. Culture supernatant (2 μg) in reducing sample buffer is loaded on a SDS-PAGE gel, along with high range rainbow molecular weight markers (GE Healthcare). Proteins are transferred from the SDS-PAGE gel to an Immobilon-P PVDF transfer membrane (Millipore) using a Mini Trans-Blot module (Bio-Rad) for one hour at seventy V. The membrane is blocked overnight with Superblock T20 PBS blocking buffer (Thermo Scientific). To detect mesothelin, blots are incubated with either Bb A1 or mAb K1 (Santa Cruz Biotechnology) at two μg/mL in Superblock for one hour at room temperature. The blots are washed three times with PBST (PBS containing 0.05% (v/v) Tween-20) and are incubated for thirty minutes with a 1:20,000 dilution of streptavidin-HRP (BD Pharmingen) in Superblock to detect Bb A1 or a 1:10,000 dilution of anti-mouse IgG HRP (GE Healthcare) in Superblock for one hour to detect mAb K1. The Ig hinge on Msln-Ig is directly detected with a 1:10,000 dilution of HRP conjugated F(ab')2 goat anti-human IgG (H+L) from Jackson Immunoresearch using a similar protocol. The blots are washed three times with PBST and detected with Luminata Classico Western HRP substrate (Millipore) using double emulsion blue basic autoradiography film (GeneMate).

Self-Assembly of Targeted Superparamagnetic Iron Oxide Nanoparticles (SPION) for Flow Cytometry

The self-assembly of immunotargeted, fluorescent nanoparticles are performed according to a previously published protocol, such as generally and specifically described by Prantner, et al., “Targeting of superparamagnetic iron oxide nanoparticles for cancer therapy based on localized hyperthermia” Jefferson Medical College, Philadelphia, Pa., 2015. Briefly, superparamagnetic iron oxide nanoparticles conjugated to streptavidin (SA-SPION) (five μL, MagCellect streptavidin ferrofluid, R&D Systems) are added to DPBS containing five mg/mL bovine serum albumin (five hundred μL, DPBS-BSA) in polystyrene round bottom tubes, mixed by vortexing, and magnetically separated using a DynaMag-2 magnet (Invitrogen) for ten minutes. The fluid is removed and replaced with YCS containing Bb A1 (five hundred μL), supplemented with fifteen ng/mL biotin-4-fluorescein (B4F, Invitrogen) for staining and ten M sodium hydroxide (2.5 μL into five hundred μL YCS) to adjust the pH. The complexes are incubated for thirty minutes at room temperature in the dark, magnetically separated for ten minutes, and washed two times with five hundred μL DPBS-bovine serum saline (BSA). After the final wash, the complexes are resuspended in DPBS containing 1% fetal calf serum for flow cytometry analysis.

Flow Cytometry Using Fluorescent SA-SPION

Ovarian cancer cell lines of human (A1847, C30) or mouse (ID8) origins are grown on tissue culture-treated plates and non-enzymatically detached by pipet mixing with a PBS-based, enzyme-free cell dissociation buffer (five mL, Gibco). Then, 10⁵ cells are incubated with the appropriate nanoparticle preparation (five hundred μL) for thirty minutes at four degrees C., washed twice with DPBS containing 1% FBS (five hundred μL, PBS-FBS) and resuspended in PBS-FBS (five hundred μL). Prior to flow cytometry, 7-amino-actinomycin D (Via-Probe, Becton Dickinson) is added to identify viable cells for subsequent analysis of the fluorescein fluorescence intensity.

Tumor Spheroid Preparation and Immunofluorescence

Tumor spheroids are generated using a liquid overlay technique modified as follows. 96-well plates are coated with 1.6% agarose (fifty μL) and allowed to solidify. Human ovarian cancer cells (A1847) are detached from a T25 flask with 0.05% trypsin/EDTA (Gibco) and resuspended in RPMI media containing 10% FBS and 1% penicillin/streptomycin at a cell density of 5×10⁵ cells/mL. Cells (two hundred μL) are applied to agarose-coated wells and maintained at thirty seven degrees C. under a humidified 5% CO2 atmosphere while rotating at one hundred twenty rpm for two days. Tumor spheroids are then washed with PBS (five hundred μL). For frozen sections, the spheroids are placed in the bottom of a cryomold, optimal cutting temperature (OCT) compound are added, and the samples are frozen on dry ice for sectioning. The sections are dried at room temperature for thirty min, fixed at room temperature for ten minutes using acetone pre-cooled to negative twenty degrees C., and then washed three times for five minutes in wash buffer (Dako). The slides are blocked for thirty minutes with serum-free protein block (Dako). Bb A1 (10 μg/mL) diluted in antibody diluent (Dako) is incubated on the slides overnight at four degrees C. in a humidified chamber. The slides are washed three times for five minutes in wash buffer before adding Alexa Fluor 488-labeled anti-V5 mAb (1:100 dilution, AbD Serotec) for one hour. Slides are counterstained with DAPI, washed three times for five minutes with wash buffer, and mounted with Fluoromount-G (SouthernBiotech). For fixed, paraffin embedded sections, the spheroids are placed in formalin for one hour, dehydrated through an ethanol gradient, and embedded in paraffin for sectioning. After mounting, slides are heated to sixty degrees C. for twenty minutes, cooled to room temperature, washed twice in xylene for fifteen minutes, rehydrated through an ethanol gradient into water. Antigen retrieval is performed using high pH antigen unmasking solution (Vector Labs). Slides are washed two times for five minutes in PBS and then once in wash buffer for five min. The slides were blocked for thirty minutes with serum-free protein block (Dako). Bb A1 (ten μg/mL) in antibody diluent (Dako) are incubated on the slides overnight at four degrees C. in a humidified chamber. The slides are washed three times for five minutes in wash buffer before adding Alexa Fluor 488-labeled anti-V5 (1:100 dilution, AbD Serotec) for one hour. Slides are counterstained with DAPI, washed three times for five minutes with wash buffer, and mounted with Fluoromount-G (SouthernBiotech). Negative controls for both the frozen and paraffin sections used the same protocol except that the slides are incubated overnight with antibody diluent instead of Bb A1. Spheroid sections are imaged with a Zeiss Axioplan upright microscope and processed using ImageJ.

Quantum dot Labelling With Cys A1

Cys-A1 can be derived from Nb A1 with standard molecular biology protocols to include a cysteine for thiol-maleimide coupling. Purified Cys-A1 is coupled to a quantum dot using a Qdot 800 antibody conjugation kit (Invitrogen) according to the manufacturer's instructions. Cells (A1847 and C30) are grown on eight-well chamber slides (Lab-Tek II CC2, Nunc) and labeled with carboxyfluorescein diacetate, succinimidyl ester (CFSE, Invitrogen) in PBS for fifteen minutes at thirty seven degrees C. Then, cells are washed, incubated for an additional thirty minutes in cell culture media, and fluorescently labeled. Cells can be first washed with five hundred pl of DPBS containing calcium and magnesium (PBS++) and blocked for non-specific binding for five minutes at four degrees C. with PBS++ supplemented with two mg/mL bovine serum albumin (BSAPBS++) (Sigma-Aldrich). Qdots labeled with Cys-A1 are diluted to either ten or fifty nM in BSA-PBS++ (two hundred μL) at four degrees C. and are added to the cells and incubated for thirty minutes at four degrees C. in the dark. Unbound Qdots are removed by aspiration and the cells can be washed three times with BSA-PBS++ (five hundred μL) at four degrees C. followed by a wash with room temperature PBS++ (five hundred μL). Cells can be mounted with Fluoromount G (Southern Biotech). The slides are imaged on an IVIS Spectrum pre-clinical in vivo imaging system (Perkin Elmer) using excitation/emission wavelengths of 500/540 nanometers (nm) for CFSE and 430/800 nm for Qdot 800.

CDR1, CDR2, and CDR3 sequences of a first positive clone (e.g., nanobodies) as previously described include CDR1 comprising SEQ ID NO:05, CDR2 comprising SEQ ID NO:06, and CDR3 comprising. SEQ ID NO:07 (e.g., nanobody A1). CDR1, CDR2, and CDR3 sequences of a second positive clone as previously described include CDR1 comprising SEQ ID NO:08, CDR2 comprising SEQ ID NO:09, and CDR3 comprising SEQ ID NO:10. Further, the isolated nanobodies and/or conjugate constructs of the isolated nanobodies, comprising the above described CDR1, CDR2, and CDR3 sequences, can specifically bind to mesothelin and/or with an affinity of 15 K_{D} and/or 30 K_{D}.

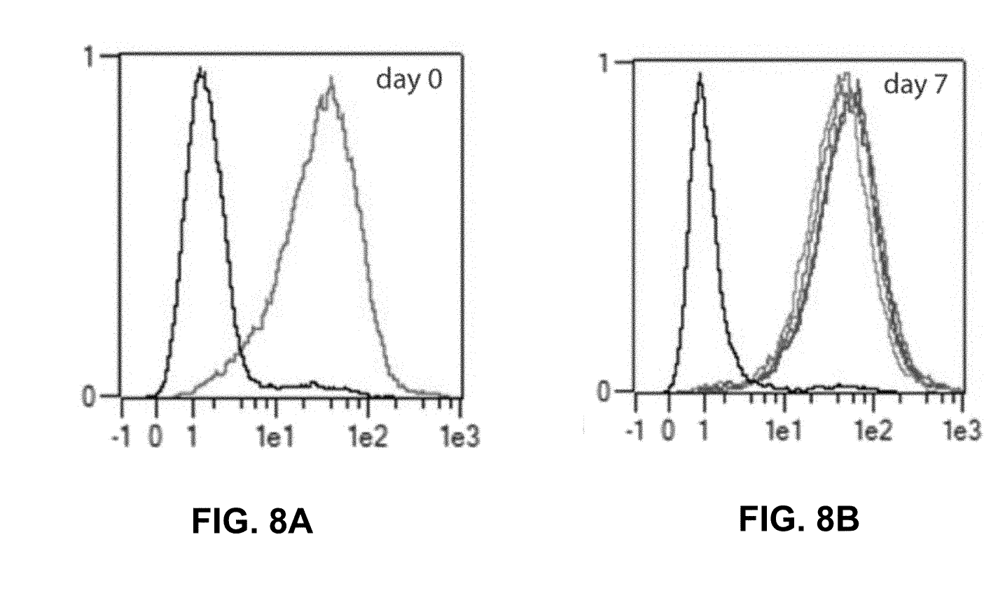

FIGS. 8A-8B illustrate examples of experimentally determined stability of nanobodies as previously described, in accordance with various embodiments. FIG. 8A illustrates the stability of Nb A1 as analyzed at day one and FIG. 8B illustrates the stability of Nb A1 at day seven. As illustrated, Nb A1 showed similar fluorescence shifts by flow cytometry compared to the initial staining after seven days at negative twenty, four, and thirty seven degrees C. in PBS or after seven days at thirty seven degrees C. in 90% human serum.

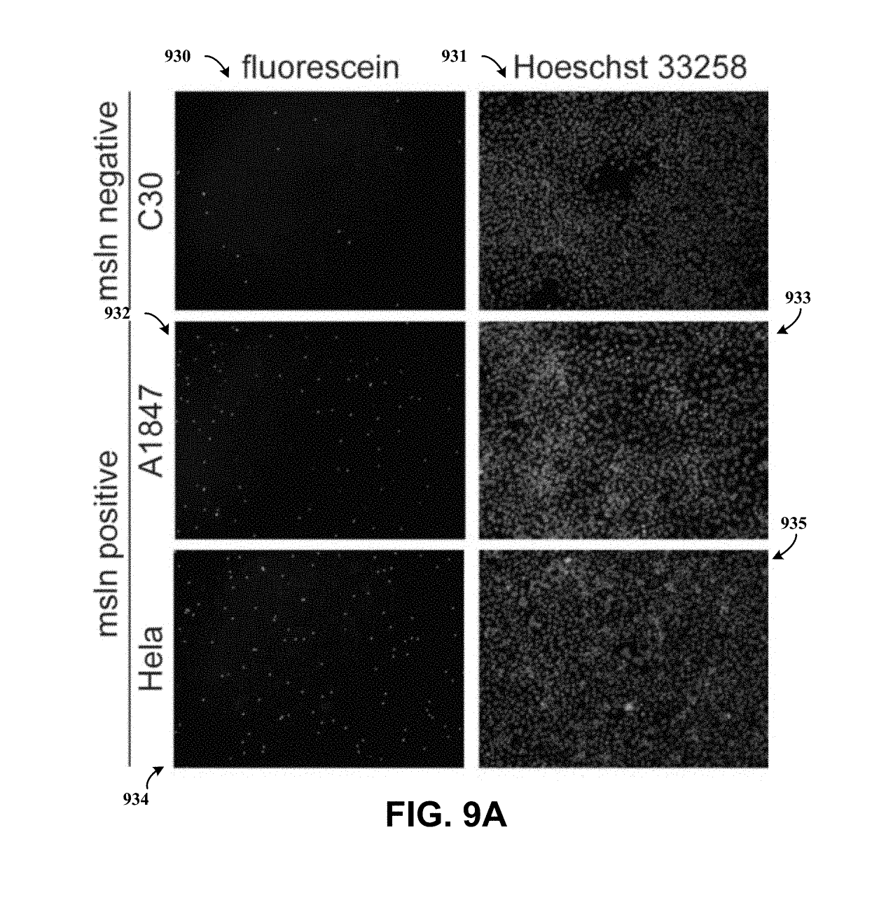

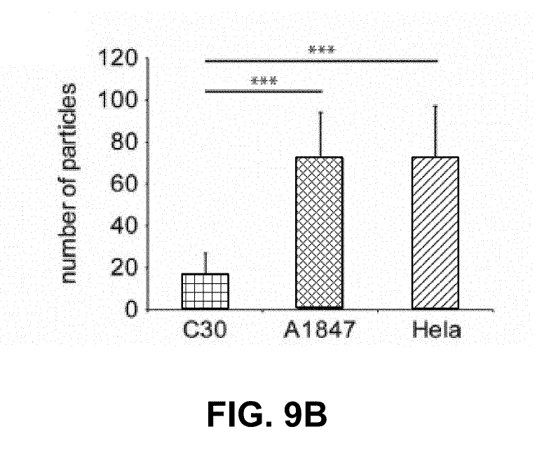

FIGS. 9A-9B illustrate examples of nanobodies binding at physiological temperature, in accordance with various embodiments. In specific experimental embodiments, to further validate the potential for Nb A1 to bind mesothelin in vivo, the nanobody specificity at thirty seven degrees C. can be determined by incubating nanobody-labeled fluorescent compensation beads with mesothelin negative (C30) and mesothelin positive (A1847 and Hela) cells for four hours. Fluorescent images show that Nb A1 can discriminate between antigen positive and antigen negative cells at thirty seven degrees C. prior to fixation and nuclear staining with Hoechst, as illustrated by FIG. 9A (e.g., images 930, 932, and 934 illustrates prior to staining with Hoechst and images 931, 933, and 935 illustrate after staining). Quantitative image analysis using ImageJ can be used to determine that the mean number and standard deviation of particles bound to C30, A1847, and HeLa cells were 17±10, 73±22, and 72±25, respectively, as illustrated by FIG. 9B.

Various experimental embodiments are directed to methods of detecting overexpression of mesothelin and/or diagnosing an organism with a condition or disease associated with overexpression of mesothelin. In specific embodiments, a human can be diagnosed with one of more of various types of cancers associated with overexpression of mesothelin, such as ovarian cancer, lung cancer, mesothelioma, breast cancer and other cancers. For example, a sample (e.g., biological sample such as blood or tissue sample) from an organism that may have cancer can be collected and the sample is placed in contact with one of the nanobodies and/or a conjugate construct thereof that specifically binds to mesothelin. In some experiment embodiments, the sample is placed in contact with a conjugate construct of Nb A1 and/or Nb C6 that specifically binds to mesothelin. The antibody (e.g., nanobody) and/or conjugate construct can be tagged with a label such that binding of the antibody and/or conjugate construct to targets present in the sample can be detected. In a specific example, an increase in detected binding of the antibody and/or conjugate construct is used to diagnose a human and/or other organism. In other embodiments, a second antibody that binds to the nanobody and/or conjugate construct (e.g., a reporter or detector, such as an anti-human antibody) can be used to detect the overexpression of mesothelin by detecting binding the second antibody to the first.

In a number of embodiments, overexpression of mesothelin in a sample can be detecting using in vivo and/or in vitro imaging. Such imaging can be used to detect human ovarian cancers and lungs cancers, although embodiments are not so limited. Various experimental embodiments include the use of mesothelin positive A1847 human ovarian cancer cells injected subcutaneously into NOD scid gamma (NSG) female mice, mesothelin negative C30 human ovarian cancer cell line, mesothelin positive EKVX human lung cancer cell line injected orthotopically into NSG female mice, mesothelin positive H460 human lung cancer cells injected orthotopically into NSG female mice, and/or mesothelin positive A549 human lung cancer injected orthotopically in NSG female mice for imaging and detection of human ovarian and lung cancers using Nb A1. Further, experiments can include the use of mesothelin positive Luc-ID8 mouse ovarian cancer cells injected orthotopically in C57BL/6 female mice, mesothelin positive Luc-ID8 mouse ovarian cancer cells injected intraperitoneally in C57BL/6 female mice, and/or mesothelin CRE-inducible ovarian cancer in Dicer/Pten^{flox/flox} female mice. The Nb A1 can recognize and bind to mesothelin. C30 ovarian cancer cell line may not express mesothelin (e.g., do not overexpress), while A1847 human ovarian cancer cell lines and Luc-ID8 mouse ovarian cancer cell lines do express (e.g., overexpress) mesothelin.

In related embodiments, in vivo imaging of the sample in contact with Nb A1 (e.g., G3a) coupled to fluorescent probe is performed. Specifically, an organism (e.g., a mouse) can receive retro-ocular injections of a labeled site-specific biotinylated, flag tagged anti-mesothelin nanobody, such as a biobody A1. The Nb can be coupled to a label, such as streptavidin IRB680W. A negative control can be used that includes the label only. Signals of the label (e.g., IRB680W) can be detected via fluorescent imaging (e.g., excitation 640 nm, emission Cy5, 1 second acquisitions). Luciferase signaling by bioluminescence can be used as a positive control (e.g., one minute acquisition).

In further specific and related embodiments, in vitro and in vivo imaging of a sample in contact with Nb A1 (e.g., G3a) coupled to magnetic beads is performed. For in vitro imaging, Nb A1 can be coupled to anti-flag magnetic beads and incubated with various cell lines before embedding the sample in agarose. Negative controls can include magnetic beads embedded in agarose and/or Nb A1 incubated with various cell lines before embedding in agarose. For in vivo imaging, the subject is provided with retro-ocular injections of the Nb A1 coupled to avidin-coated magnetic beads. A negative control can include retro-ocular injections of the subject with avidin-coated magnetic beads only. A subject, as used herein, includes an organism being imaged and/or testing, such as for diagnosis or experimental purposes.

In a specific experimental embodiment, in vivo magnetic resonance imaging (MRI) imaging with anti-mesothelin nanobodies is performed in a mouse model of ovarian cancer. Recombinantly expressed nanobodies that recognize mesothelin are site-specifically biotinylated using a biosynthetic protocol. Immunotargeted nanoparticles are prepared by incubating anti-mesothelin nanobodies (e.g., thirty ug) with streptavidin-coated iron oxide nanoparticles (fifty ug, Nanoc) for thirty minutes. Mice are initially anesthetized by inhalation of 2% isofluorane in 100% oxygen and anesthesia is maintained throughout the MRI data collection using 1.5% isofluorane. The mice have topical Puralube vet ointment applied to their eyes and respiration is monitored during MRI data collection, such as using a Model 1030-S-50 Small animal monitoring and gating system (SA Instruments, Inc.). Coronal images of mice are collected before and after receiving retro-orbital injections of nanoparticles labeled with anti-mesothelin nanobodies. The MRI data is collected on a sixteen centimeter horizontal bore Bruker PharmaScan 70/16 using Paravision 6.0.1 with a forty millimeter (mm) coil at pre injection, one hour, four hours and twenty-four hours post injection of nanobodies. Images are collected, such as using a T2 TurboRare sequence with a 25×25 mm field of view (FOV), image size 28×28, slice thickness=0.5 mm, TR=2400 ms, 20 slices, TE=37 ms, RARE factor=4, an effective echo time=18.5 ms with a total scan time of 0 h 5 m 7 s 200 ms. A FLASH sequence with a 25×25 mm FOV, slice thickness=0.5 mm, 5 averages, TR=230.00 ms, TE=2.3 ms, 20 slices with a scan time of 0 h 2 m 27 s 00 ms, although embodiments are not so limited. For more general and specific information related to biobodies and site-specifically biotinylation using a biosynthetic protocol, reference is made to U.S. Pat. No. 7,795,411, entitled “Vectors For Expressing in Vivo Biotinylated Recombinant Proteins”, filed Jan. 31, 2007, and to Scholler, et al., “Method for generation of in vivo biotinylated recombinant antibodies by yeast mating”, J Immunol Methods 317(1-2): 132-143 (2006), each of which is fully incorporated herein for their teaching.

The above-described experimental embodiment for in vivo detection of overexpression of mesothelin can be extended to human applications through some modifications. For example, incorporating a cysteine residue to the nanobody (e.g., Nb1 or C6) at a location that is not sterically hindered can allow for site-specific and homogeneous modification of the nanobody with small molecule drugs or imaging agents. The lysine residue in the biotinylation sequence is an ideal candidate for mutation to a cysteine because it is in a region that is accessible to the biotinylation enzyme. The cysteine bioconjugates could be used for various diagnostic, prognostic, and therapeutic applications in precision medicine. For example, a nanobody drug conjugate can modify the biodistribution of a cytotoxic drug, and a conjugate with indocyanine green (a Food and Drug Administration (FDA) approved near-infrared fluorophore) can provide an antigen-specific near-infrared dye for fluorescence-guided surgery. The thiolate-mediated bioconjugation of the nanobody can couple the nanobody directly to a thiol reactive functional group or modify the cysteine to an azide using a heterobifunctional crosslinker to provide a site for click chemistry.

The immunogenicity of single domain antibodies (e.g., Nbs) and the induction of neutralizing antibodies can be a concern and/or can be tested for, such as when using a xenoprotein. Prior to human translation, a preclinical assessment of the nanobody immunogenicity can be performed using a combination of computer predictions and experimental validation. If the construct is immunogenic, the nanobody can be humanized by either grafting the antigen binding loops onto a general humanized scaffold or minimally humanizing the nanobody by humanizing the residues in framework regions 1, 3, and 4 which have minimal effect on binding affinity. The resulting construct can be evaluated for binding affinity prior to analysis of immunogenicity risk. For more general and specific information related to preclinical assessment techniques and humanizing antibodies and nanobodies, reference is made to: King et al., “Removing T-cell epitopes with computational protein design,” Proc Natl Acad Sci USA, 111(23): 8577-8582 (2014); Mazor, et al., “Recombinant immunotoxin for cancer treatment with low immunogenicity by identification and silencing of human T-cell epitopes”, Proc Natl Acad Sci USA, 111(23): 8571-8576 (2014); and Vincke, et al., “General strategy to humanize a camelid single-domain antibody and identification of a universal humanized nanobody scaffold”, J Biol Chem, 284(5): 3273-3284 (2009), each of which are fully incorporated herein for their teachings.

In further specific experimental embodiments, for in vitro MRI imaging, the Nb (e.g., twenty-five ug/ml of Nb A1) is pre-incubated with magnetic beads at various ratios for at least thirty minutes at four degrees C. The magnetic beads can be coated with anti-flag or avidin. Tumor cells lines, such as C30, IDB, and A1847, are stained for thirty minutes with the Nb-magnetic bead complexes at four degrees C. The cells from the sample are fixed with two percent PFA for twenty minutes at four degrees C. Following, the cells are resuspended in one hundred ul 1×PBS. A phantom tube with two percent ultralow gel temperature agarose cell as spacers and cells in one percent agarose gel is created.

For in vivo MRI imaging, in some embodiments, the Nb (e.g., thirty ug/ml or twenty-five ug/ml of Nb A1) is pre-incubated with magnetic beads that are coated with anti-flag or avidin for at least thirty minutes at four degrees C. The avidin or anti-flag coated magnetic beads can be fifty ug/ml, in various embodiments. An organism, such as a mouse (or other subject), receives intravenous (IV) injections of the Nb-magnetic bead complexes via retro orbital injection. In specific experimental embodiments, the IV injection includes one hundred to one hundred-fifty ul of the Nb-magnetic bead complexes. The organism is imaged before and after the injection, such as periodically at one hour, four hours, and twenty-four hours. The images (e.g., Image J) are used to quantify signal intensities by creating regions of interest (ROI) of an area of interest (e.g., an area, organ, or tissue suspected to be or have a tumor) and of control tissue (e.g., muscle) across each slice. Signal intensity (SI) is calculated by multiplying the area of the ROI and signal intensity and SI of the ROI is normalized by SI of the tumor or muscle (e.g., control tissue).

For in vivo imaging system (IVIS) imaging, the Nb (e.g., thirty ug/ml of Nb A1) is pre-incubated with labeled streptavidin IRB680W (1:1) for at least thirty minutes on ice to form complexes. The organism, such as a mouse (or other subject), receive IV injections of eighty to one hundred-twenty ul of the Nb-labeled streptavidin complexes via retro orbital injection. The animal is imaged via IVIS. For IRB680W, fluorescent signals can be recorded before and after the injection at various time periods. Organisms bearing luciferase-transduced tumors (e.g., Luc-ID8) can also be imaged by bioluminescence once as a positive control of tumor burden.

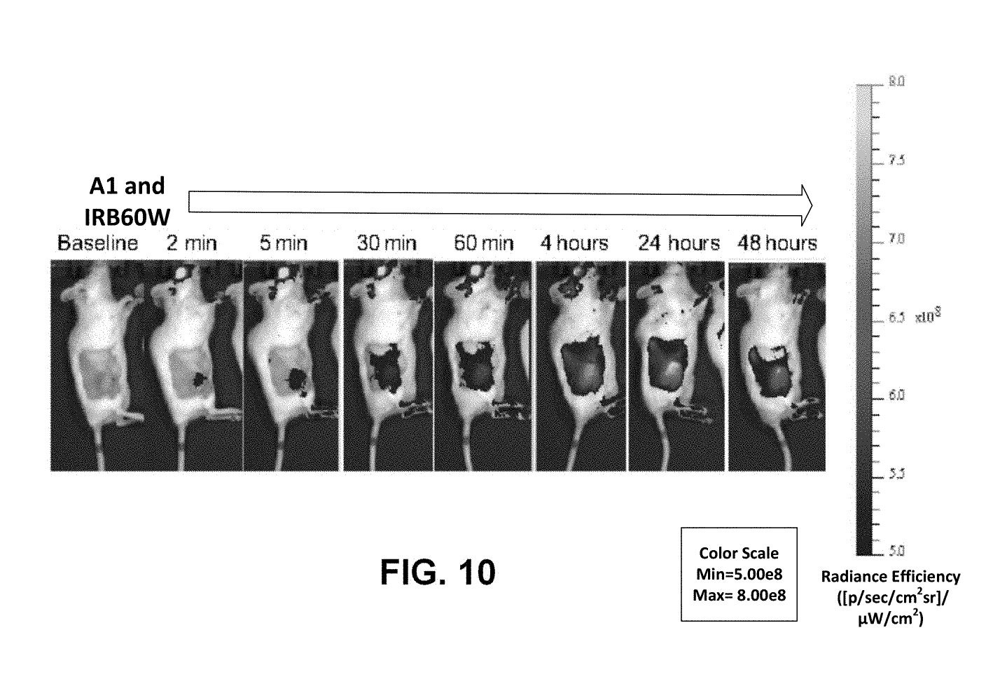

FIG. 10 illustrates example images of a mouse treated with human mesothelin human ovarian cancer cell line and an antibody, in accordance with various embodiments. Specifically, the Nb A1 is combined with streptavidin, as described above, and used to detect mesothelin-expresser ovarian cancer cells by in vivo imaging (IVIS). The mouse illustrated by FIG. 10 is a NSG mouse injected subcutaneously with human mesothelin positive A1847 human ovarian cancer cell lines and the nanobody-streptavidin complex. The mouse, as illustrated, includes subcutaneous tumors. The tumors can be detected via imaging of the mouse prior the injection and periodically after the injection of the nanobody-streptavidin complex. The signal intensity is detected, which can be based on the scale illustrated on the right side of FIG. 10.

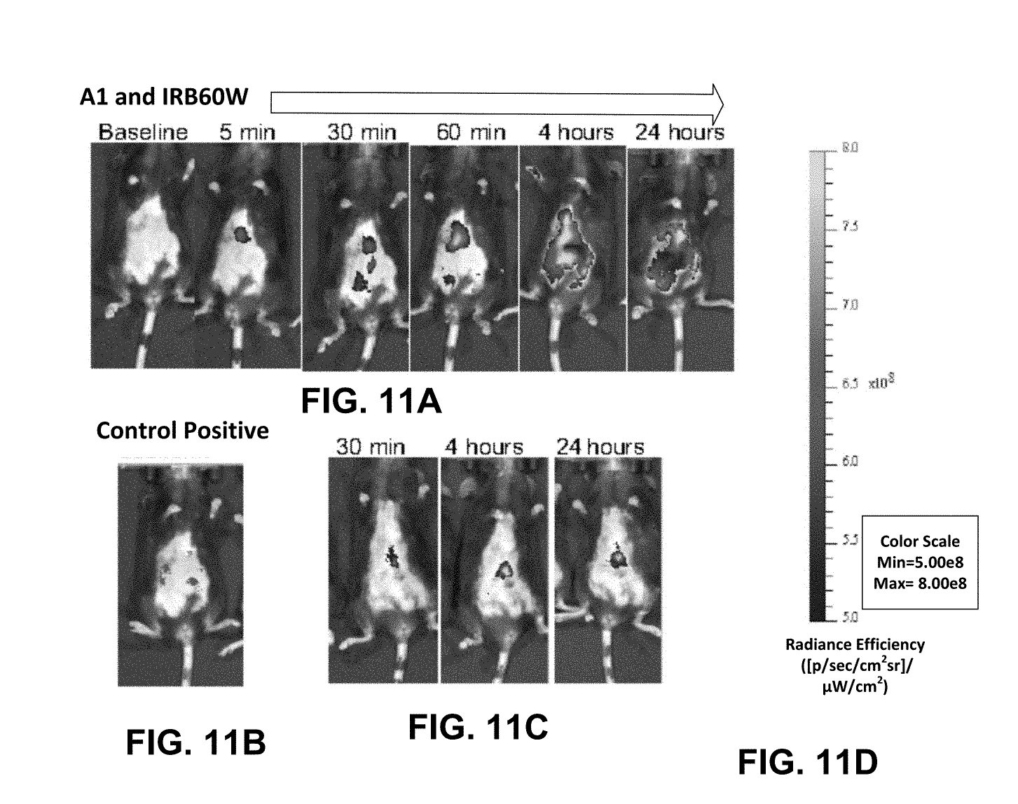

FIGS. 11A-11D illustrate example images of a mouse treated with mouse mesothelin ovarian cancer cell line and an antibody, in accordance with various embodiments. The mice illustrated by FIGS. 11A-11C can include C57BL/6 mice. FIG. 11A illustrates images of a test mouse. The test mouse can be injected (peritoneally) with Luc-ID8 ovarian cancer cell lines and with the Nb A1-streptavidin complex. Images are illustrated prior to the injection and periodically after the injection. FIG. 11B illustrates an example positive control mouse. The positive control mouse bears luciferase-transduced tumors (e.g., Luc-ID8) and is imaged by bioluminescence once for a positive control of tumor burden. FIG. 11C illustrates an example of a negative control mouse. The negative control mouse is injected with streptavidin only and imaged after the injection periodically. FIG. 11D illustrates an example scale for signal intensity of the images.

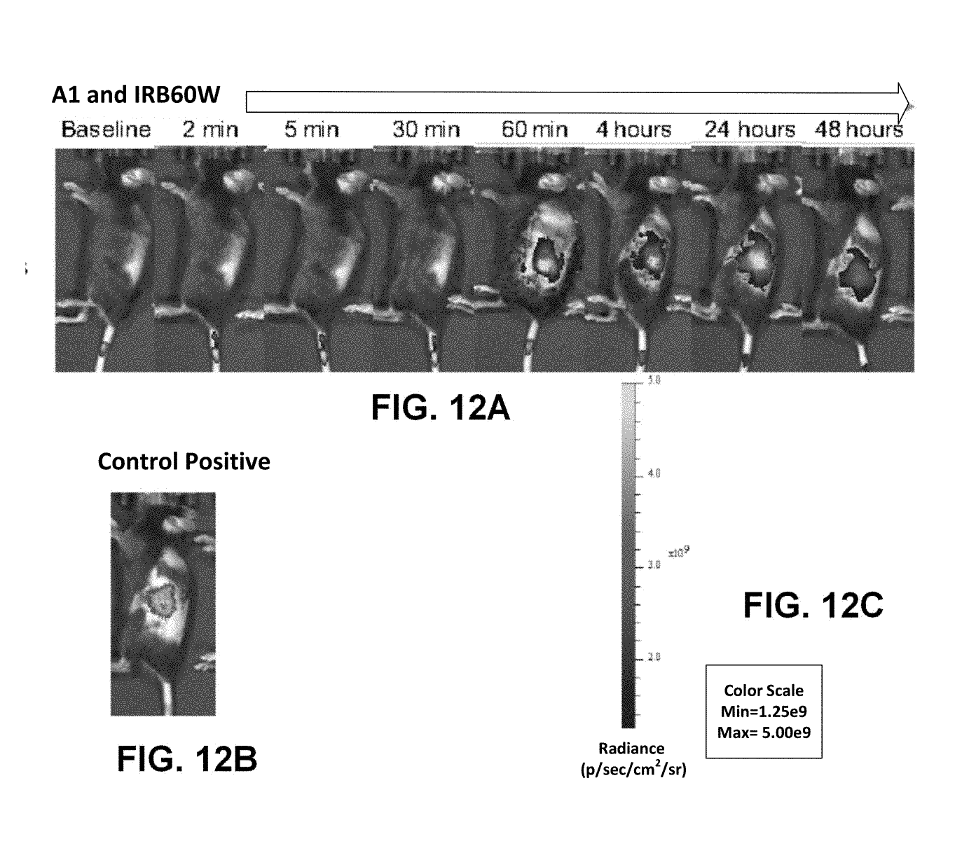

FIGS. 12A-12C illustrate example images of a mouse treated with mouse mesothelin ovarian cancer cell line and an antibody, in accordance with various embodiments. The mice illustrated by FIGS. 12A-12B can include C57BL/6 mice. FIG. 12A illustrates images of a test mouse. The test mouse can be injected (intraovarianally) with Luc-ID8 ovarian cancer cell lines and with the Nb A1-streptavidin complex. Images are illustrated prior to the injection and periodically after the injection. FIG. 12B illustrates an example positive control mouse. The positive control mouse bears luciferase-transduced tumors (e.g., Luc-ID8) and is imaged by bioluminescence once as a positive control of tumor burden. FIG. 12C illustrates an example scale for signal intensity of the images.

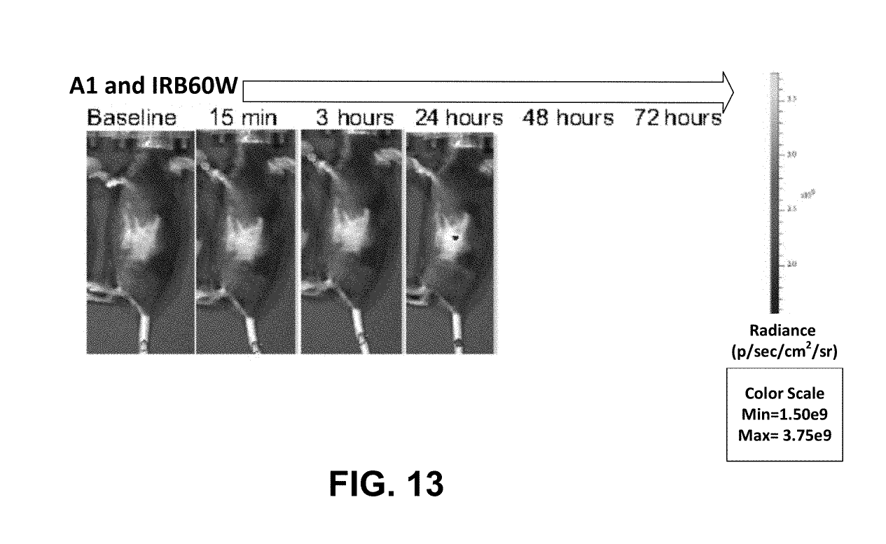

FIG. 13 illustrates example images of a mouse having no tumor that is treated with an antibody, in accordance with various embodiments. The mouse can be injected with the Nb A1-streptavidin complex. Images of the mouse are taken before the injection and periodically after. On the right side of FIG. 13 illustrates an example scale for signal intensity of the images.

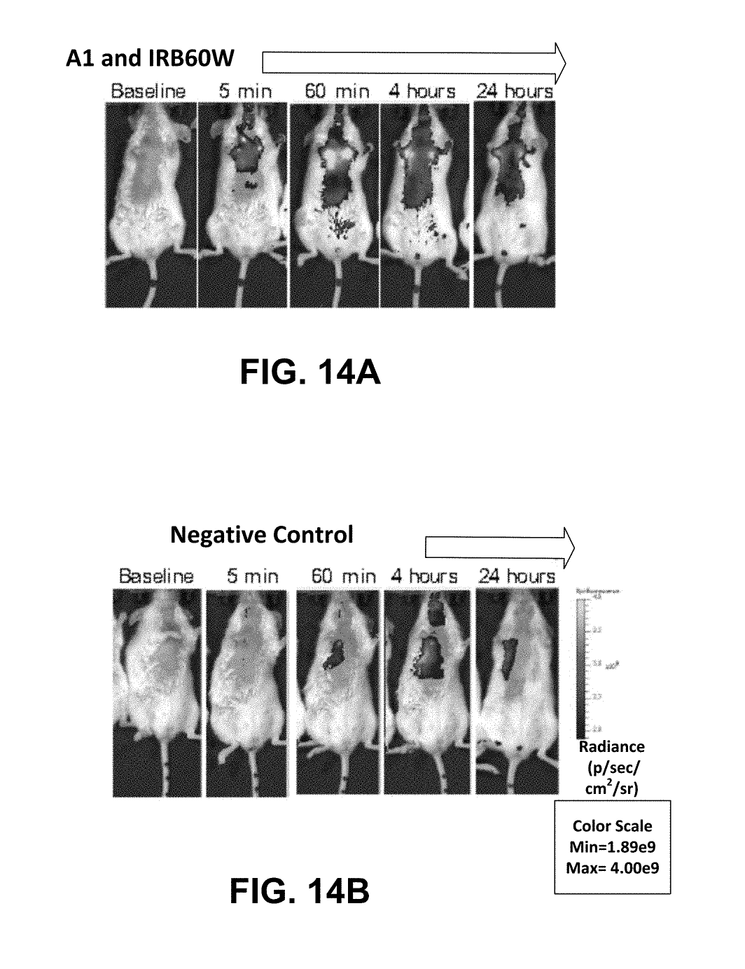

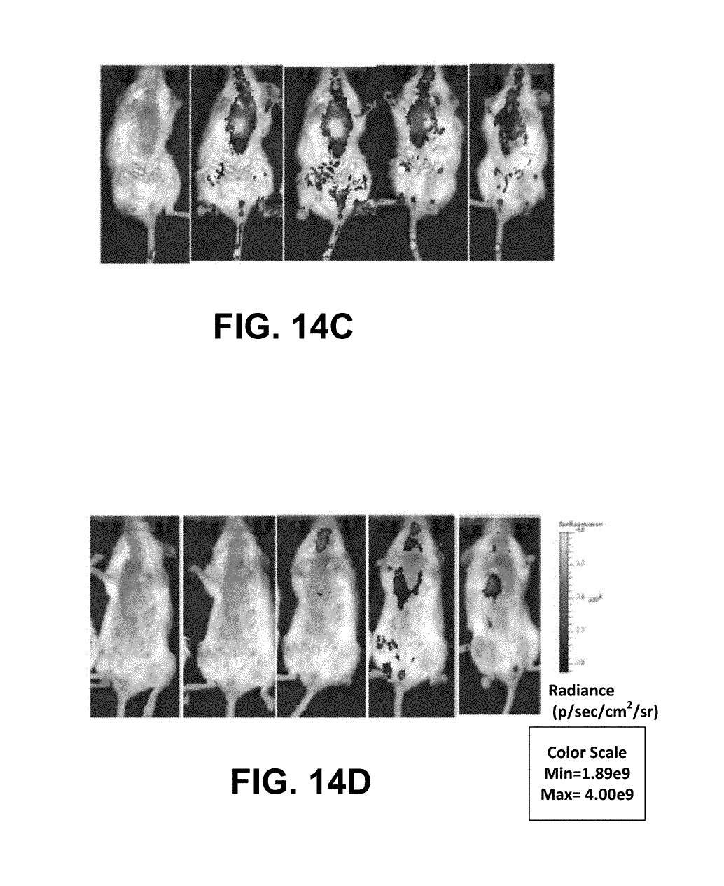

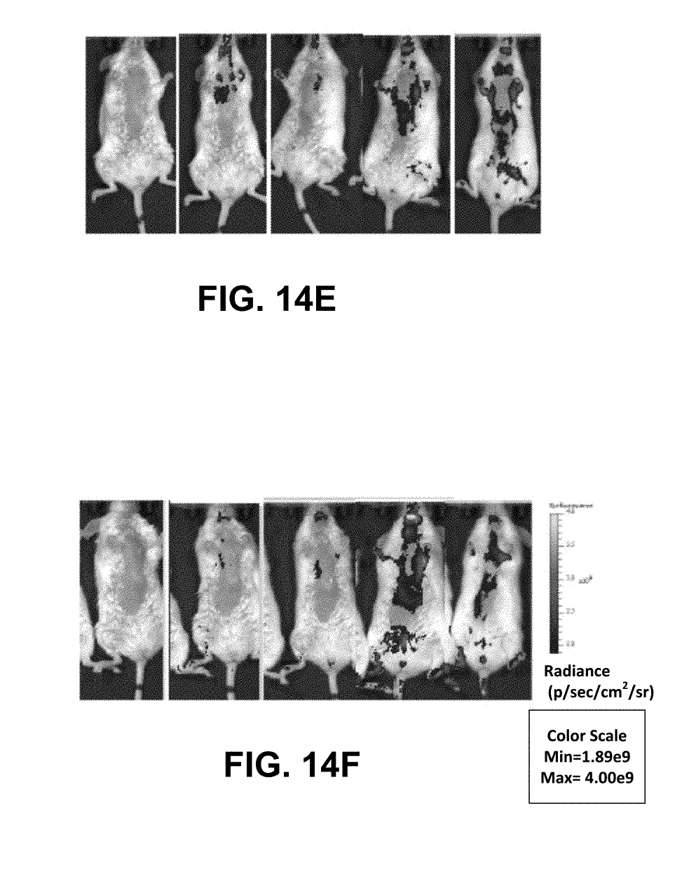

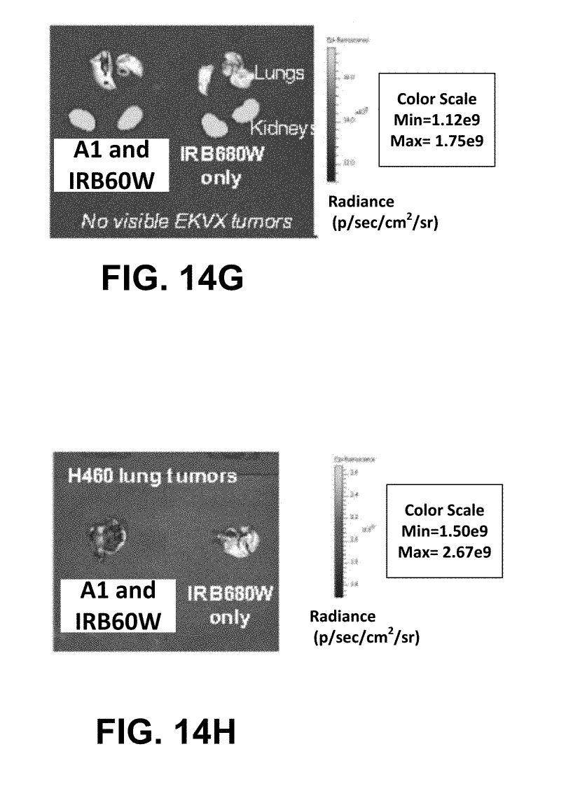

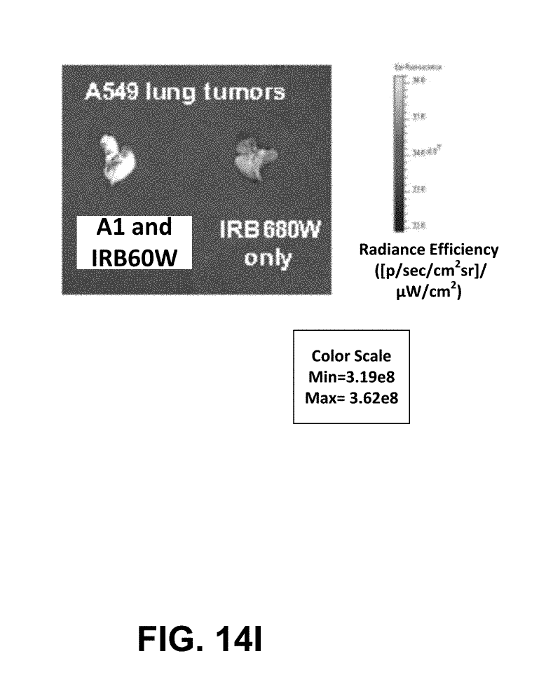

FIGS. 14A-14I illustrate example images of mice treated with mesothelin human lung cancer cell line and an antibody, in accordance with various embodiments. Test mice are injected with various forms of human lung cancer cell lines, including EKVX, H460, and A549, each of which are mesothelin positive cancer cell lines. Specifically, EKVX can include a high expression of mesothelin relative to H460 and A549. H460 can express mesothelin at a lower rate than EKVX and at a higher rate than H460. And, H540 can express mesothelin at a lower rate than both EKVX and H460. The negative control mice are injected with streptavidin only and imaged before the injection and after the injection periodically.

FIG. 14A illustrates an example of a test mouse injected with mesothelin-positive EKVX human lung cancer cell lines followed by injection with Nb A1-streptavidin complex. The mouse is imaged before the injection and periodically after the injection. FIG. 14B illustrates and example negative control mouse.

FIG. 14C illustrates an example of a test mouse injected with mesothelin-positive H460 human lung cancer cell lines followed by injection with Nb A1-streptavidin complex. The mouse is imaged before the injection and periodically after the injection. FIG. 14D illustrates and example negative control mouse.

FIG. 14E illustrates an example of a test mouse injected with mesothelin-positive A549 human lung cancer cell lines followed by injection with Nb A1-streptavidin complex. The mouse is imaged before the injection and periodically after the injection. FIG. 14F illustrates and example negative control mouse.

FIGS. 14G-14I illustrate example images of the organs of the test mice illustrated by FIGS. 14A, 14C, and 14E respectively. Each mouse organ includes the Nb A1-streptavidin complex and streptavidin only. As illustrated, no tumors may be detected.

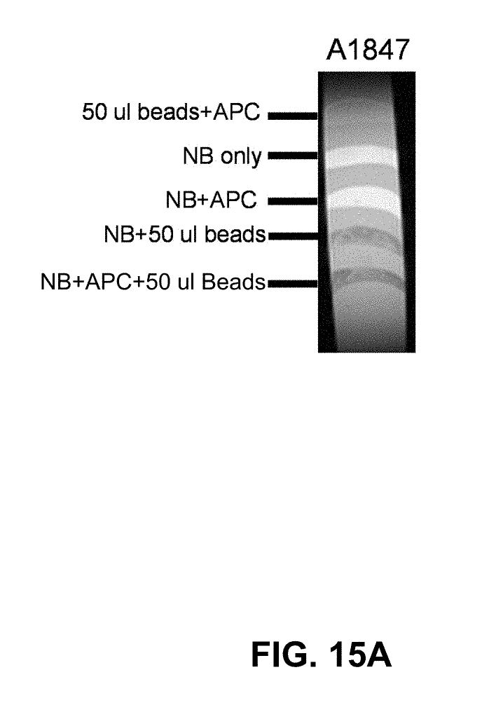

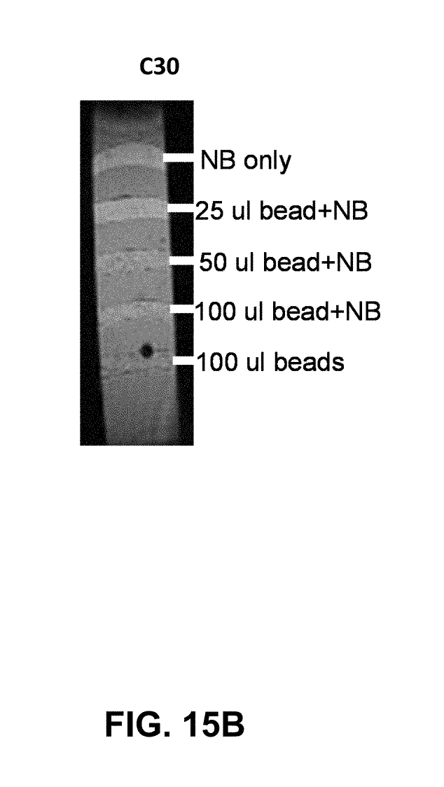

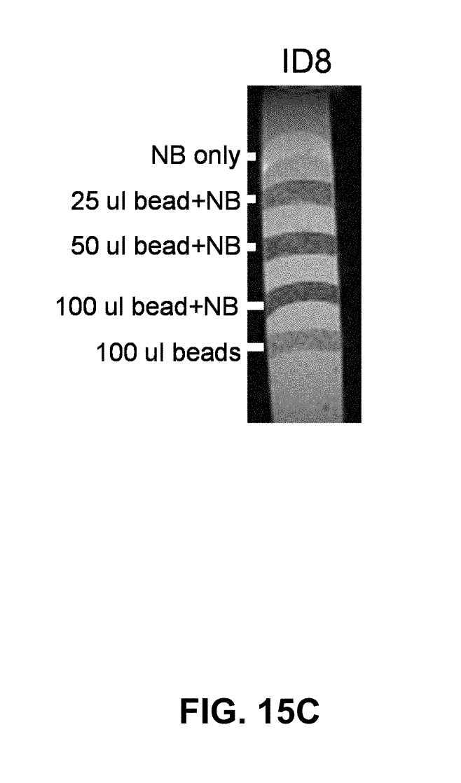

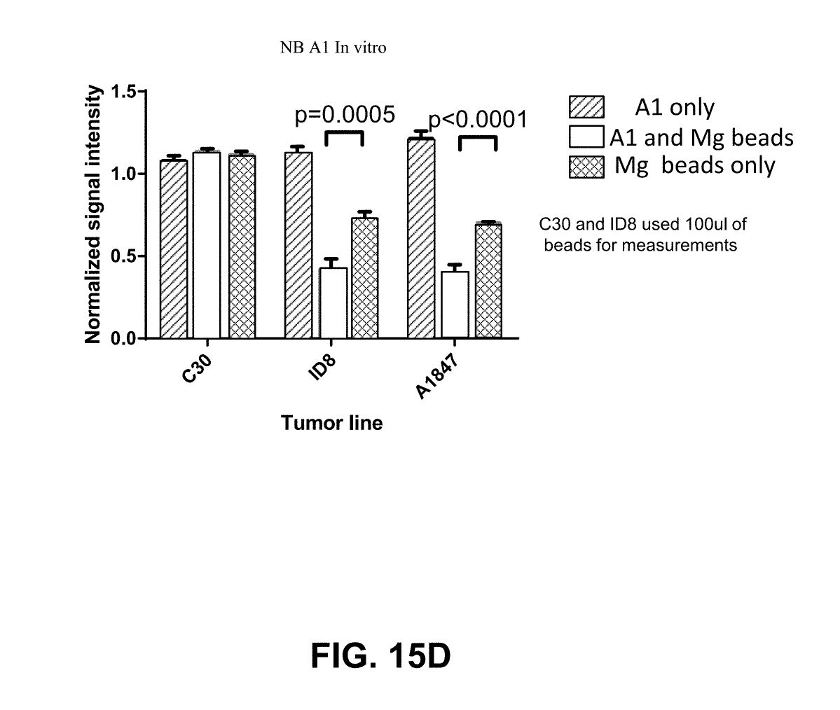

FIGS. 15A-15D illustrate examples of detecting mesothelin-expresser ovarian cancer cells in vitro using an antibody, in accordance with various embodiments. In vitro imaging of a sample can be used to detect mesothelin-expressing ovarian cancer cells. The ovarian cancer cells, in experimental embodiments, include A1847 human ovarian cancer cell line, C30 human ovarian cancer cell line, and ID8 mouse ovarian cancer cell line. A1847 can include a high expression of mesothelin relative to C30 and ID8. ID8 can express mesothelin at a lower rate than A1847 and at a higher rate than C30. And, C30 can express mesothelin at a lower rate than both A1847 and ID8. FIG. 15A illustrates an example of image obtained in vitro of a phantom tube created using Nb A1-magnetic beads complexes and the cell line A1847. As illustrated, the image confirms that Nb A1 can be used to detect expression of A1847. FIG. 15B illustrates an example image obtained in vitro of a phantom tube created using Nb A1-magnetic beads complexes and the cell line C30. FIG. 15C example of image obtained in vitro of a phantom tube created using Nb A1 magnetic beads complexes and the ID8. FIG. 15D illustrates normalized signal intensities of each of the tumor cell lines experimentally tested.

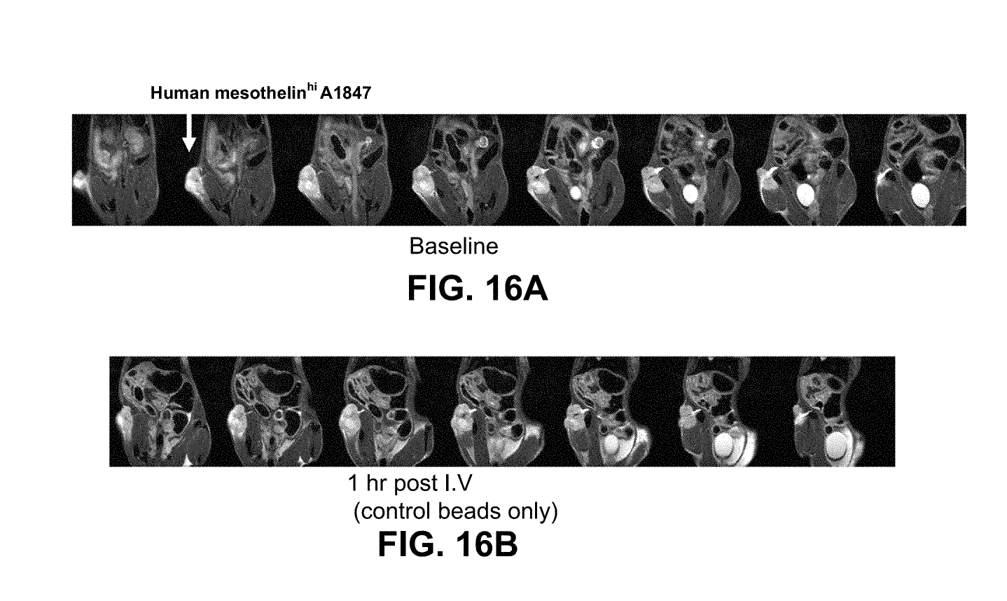

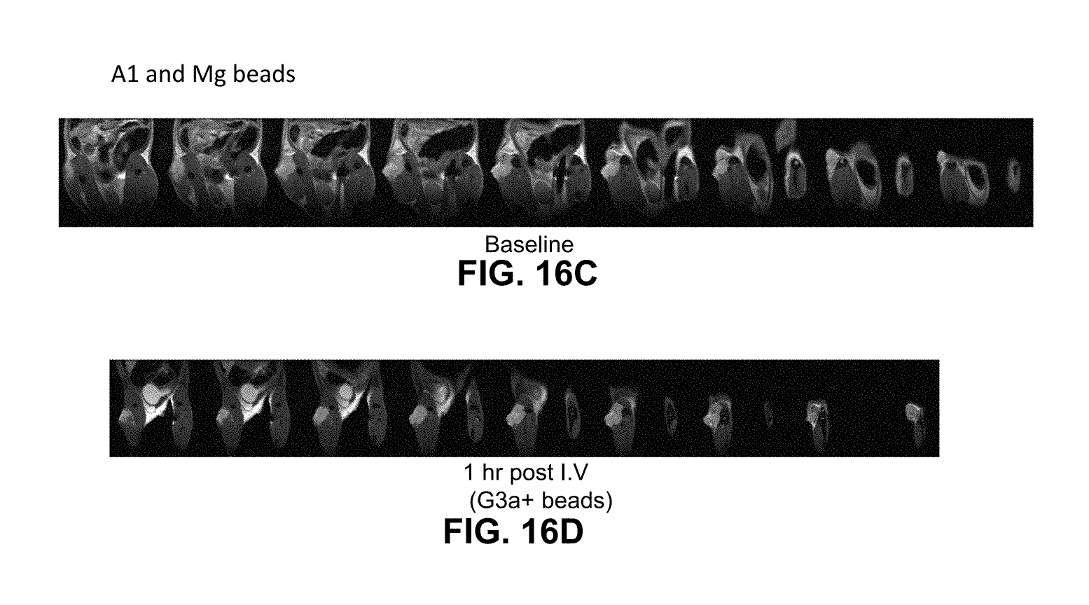

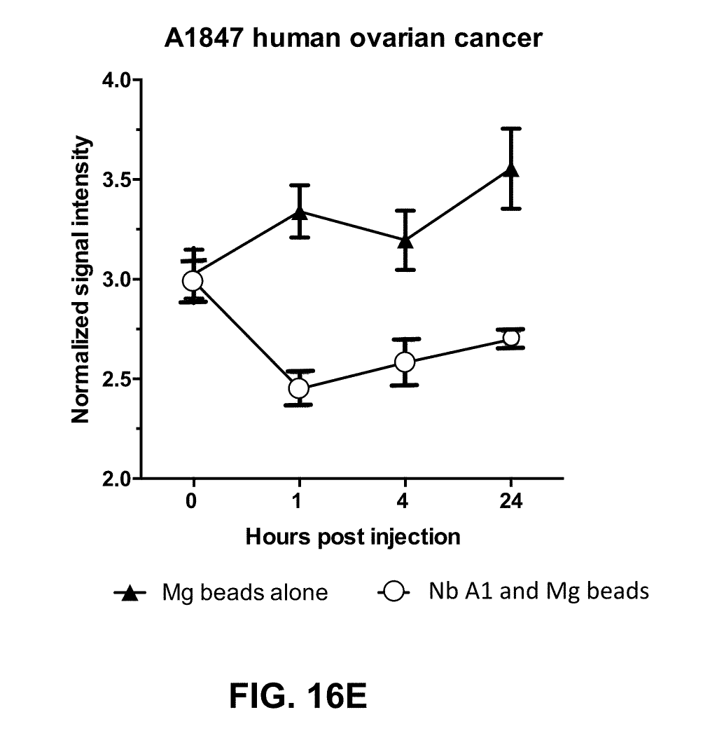

FIGS. 16A-16E illustrate example images of detection of human ovarian cancer cells using an antibody, in accordance with various embodiment. Specifically, as described above, a Nb (e.g., Nb A1) is combined with avidin (or anti-flag)-coated magnetic beads and used to detect human ovarian cancer cells by MRI imaging. A test mouse, in specific experimental embodiments, can be injected with mesothelin-expressing A1847 human ovarian cancer cell lines (subcutaneously) and the Nb-coated magnetic bead complexes. A control mouse can be injected with the magnetic beads only. Images can be taken before and after imaging to provide a base line and post IV injection. FIG. 16A illustrates an MRI image of the control mouse taken as a baseline and FIG. 16B illustrates MRI images of the control mouse one hour after injecting the control mouse. FIG. 16C illustrates MRI images of the test mouse taken as a baseline and FIG. 16D illustrates MRI images of the test mouse one hour after injection. FIG. 16E illustrates the signal intensity results of the test mouse and the control mouse, and confirms the MRI detected binding of the anti-mesothelin nanobody to mesothelin expressing tumors of ovarian cancer (e.g., A1847).

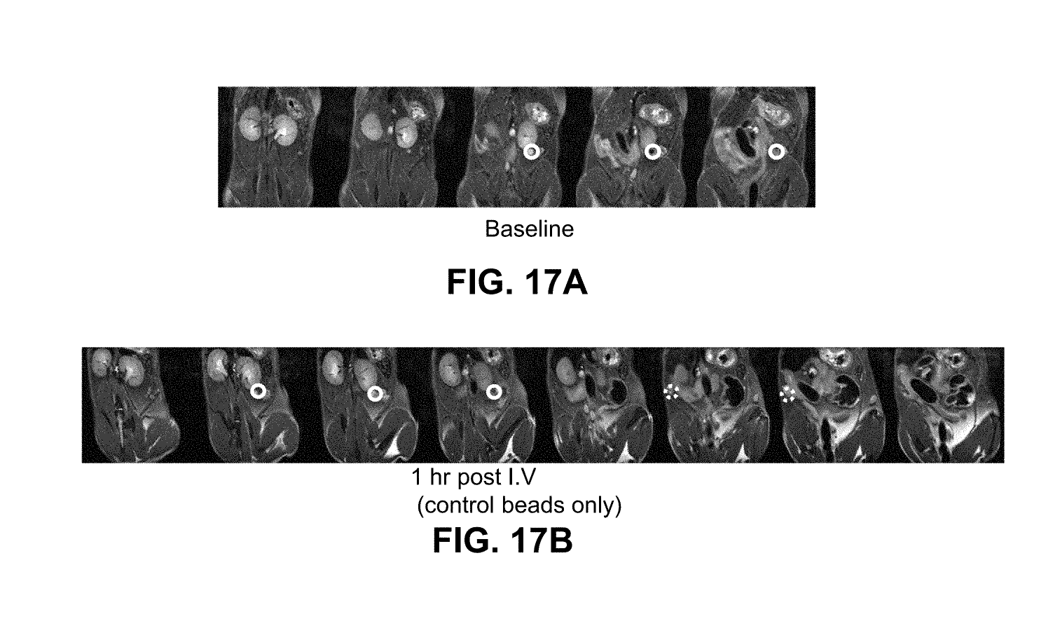

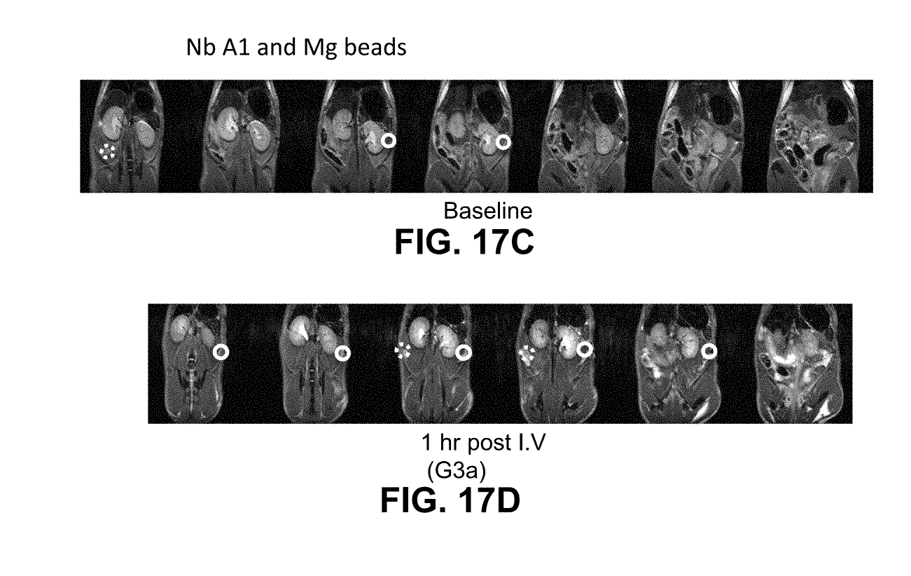

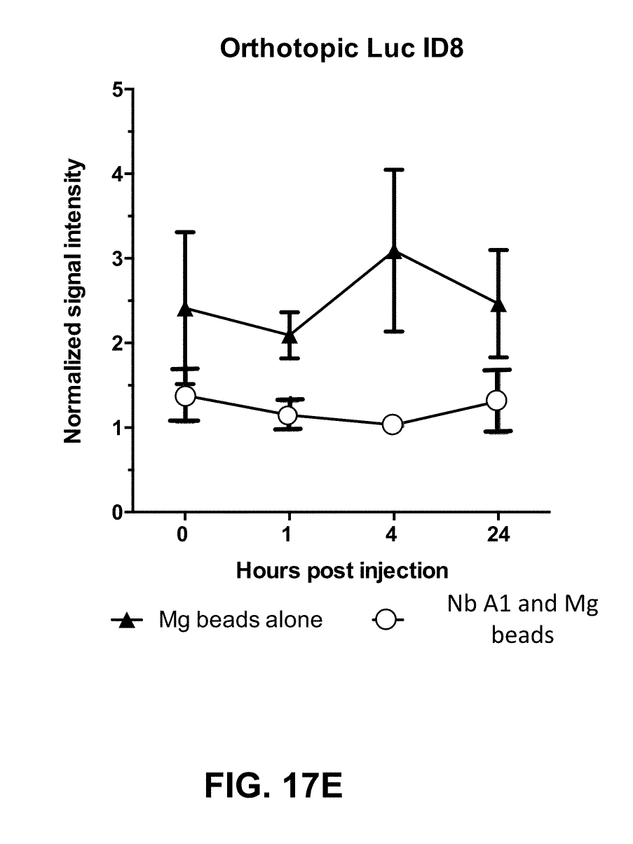

FIGS. 17A-17E illustrate example images of detection of mouse ovarian cancer cells using an antibody, in accordance with various embodiment. As described above, a Nb (e.g., A1) is combined with avidin (or anti-flag)-coated magnetic beads and used to detect mouse ovarian cancer cells by MRI imaging. The test mouse, in specific experimental embodiments, can be injected with mesothelin-expressing Luc-ID8 mouse ovarian cancer cell lines (injected intra-ovary) and the nanobody and avidin-coated magnetic bead complex. A control mouse can be injected with the magnetic beads only. Images can be taken before and after imaging to provide a base line and post IV injection. FIG. 17A illustrates an MRI image of the control mouse taken as a baseline and FIG. 17B illustrate MRI images of the control mouse one hour after injecting the control mouse. FIG. 17C illustrates MRI images of the test mouse taken as a baseline and FIG. 17D illustrates MRI images of the test mouse one hour after injection. The white circles illustrated in FIGS. 17A-17D highlight the tumor injected ovary (and also illustrate the detection of overexpression of mesothelin) and the white-dashed circle illustrate the normal ovary. FIG. 17E illustrates the signal intensity results of the test mouse and the control mouse and confirms the MRI detected binding of the anti-mesothelin nanobody to mesothelin expressing tumors in a mouse model of ovarian cancer (e.g., Luc-ID8).

As described above, the example embodiments are directed to an antibody (e.g., nanobody), a conjugate construct of the antibody, and/or a composition having an antibody domain that specifically binds to mesothelin, such as human mesothelin. The antibody (e.g., nanobody), a conjugate construct of the antibody, and/or a composition having an antibody domain can bind to mesothelin for use in a method of diagnosis and/or treating a condition associated with overexpression of mesothelin. The antibody (e.g., nanobody), a conjugate construct of the antibody, and/or a composition having an antibody domain can comprise a variable region comprising SEQ ID NO:02 and/or SEQ ID NO:04. In specific embodiments, the antibody (e.g., nanobody), a conjugate construct of the antibody, and/or a composition having an antibody domain comprising CDRs selected from the group consisting of SEQ ID NO:05, SEQ ID NO:06, SEQ ID NO:07, SEQ ID NO:08, SEQ ID NO:09, and SEQ ID NO:10. In further specific embodiments, the antibody (e.g., nanobody), a conjugate construct of the antibody, and/or a composition having an antibody domain comprising CDR1 of SEQ ID NO:05, CDR2 of SEQ ID NO:06, and CDR3 of SEQ ID NO:07 or CDR1 of SEQ ID NO:08, CDR2 of SEQ ID NO:09, and CDR3 of SEQ ID NO:10. In various embodiments, the above described sequences are modified to form conjugate constructs of the nanobodies, as described herein. The antibody (e.g., nanobody), a conjugate construct of the antibody, and/or a composition can include a camelid and/or conjugate form thereof that binds to mesothelin with an affinity of 15 nM or 30 nM.

In various specific embodiments, the cDNA sequence of Nb A1 is provided by SEQ ID NO:01. SEQ ID NO:02 provides an example amino acid sequence of the variable region of Nb A1. The cDNA sequence of Nb C6 can be provided by SEQ ID NO:03. SEQ ID NO:04 provides an example amino acid sequence of the variable region of Nb C6. In accordance with specific embodiments, the Nb A1 comprises a CDR1 comprising SEQ ID NO:05, a CDR2 comprising SEQ ID NO:06, and a CDR3 comprising SEQ ID NO:07. In other specific embodiments, the Nb C6 comprises a CDR1 comprising SEQ ID NO:08, a CDR2 comprising SEQ ID NO:09, and. a CDR3 comprising SEQ ID NO:010. Further, the Nb disclosed herein can bind to (human) mesothelin.

Although the embodiments illustrated by the various experimental embodiments describe use of nanobody to image and detect the presence of overexpression of mesothelin in a mouse, embodiments are not so limited. For example, in various embodiments, nanobody and/or conjugate constructs can be used to identified and used for treatment of other organisms, such as humans, dogs, cats, birds, horses and other vertebrate. Terms to exemplify orientation, such as on top, onto, within, may be used herein to refer to relative positions of elements as shown in the figures. It should be understood that the terminology is used for notational convenience only and that in actual use the disclosed structures may be oriented different from the orientation shown in the figures. Thus, the terms should not be construed in a limiting manner.

Various embodiments are implemented in accordance with the underlying Provisional Application (Ser. No. 62/308,883), entitled “Anti-Mesothelin Nanobodies”, filed Mar. 16, 2016, to which benefit is claimed and is fully incorporated herein by reference. For instance, embodiments herein and/or in the provisional application (including the appendices therein) may be combined in varying degrees (including wholly). Reference may also be made to the experimental teachings and underlying references provided in the underlying provisional application, each of which are fully incorporated herein for their specific and general teachings related to antibodies, nanobodies, mesothelin, among other teachings. As a specific example, amino acid and nucleic acid sequences of mesothelin used herein and the various nanobodies and constructs thereof specifically bind to can be determined from the MSLN gene transcript found at NCBI accession number NM 005823 or NCBI accession number NM 013404. The mesothelin can include the human mesothelin or epitope of the human mesothelin. For more specific and general information related to metholethin and sequences comprising the same, reference is made to Scholler N, et al., “Development of a CA125-mesothelin cell adhesion assay as a screening tool for biologics discovery”, Cancer Lett, 247(1):130-6 (2007), which is herein fully incorporated by reference. Embodiments discussed in the Provisional Application are not intended, in any way, to be limiting to the overall technical disclosure, or to any part of the claimed invention unless specifically noted.

Various embodiments described above, and discussed provisional application may be implemented together and/or in other manners. One or more of the items depicted in the present disclosure and in the underlying provisional application can also be implemented separately or in a more integrated manner, or removed and/or rendered as inoperable in certain cases, as is useful in accordance with particular applications. In view of the description herein, those skilled in the art will recognize that many changes may be made thereto without departing from the spirit and scope of the present disclosure.

What is claimed is:

1. An isolated nanobody, a conjugate construct of the isolated nanobody, or an antigen-binding domain fragment thereof, that specifically binds to mesothelin, comprising:
a complementary determining region (CDR)1 comprising sequence SEQ ID NO:05 or sequence SEQ ID NO:08;
a CDR2 comprising sequence SEQ ID NO:06 or sequence SEQ ID NO:09; and
a CDR3 comprising sequence SEQ ID NO:07 or sequence SEQ ID NO:10.

2. The isolated nanobody, a conjugate construct of the isolated nanobody, or an antigen-binding domain fragment thereof of claim 1, comprising:
the CDR1 comprising the sequence SEQ ID NO:05, the CDR2 comprising the sequence SEQ ID NO:06 and the CDR3 comprising sequence SEQ ID NO:07, or
the CDR1 comprising the sequence SEQ ID NO:08, the CDR2 comprising the sequence SEQ ID NO:09 and the CDR3 comprising the sequence SEQ ID NO:10.

3. The isolated nanobody, conjugate construct of the isolated nanobody, or antigen-binding domain fragment thereof of claim 1, having a variable sequence comprising an amino acid sequence SEQ ID NO:02 or amino acid sequence SEQ ID NO:04.

4. The isolated nanobody, conjugate construct of the isolated nanobody, or antigen-binding domain fragment thereof of claim 1 that specifically binds to an epitope of human mesothelin.

5. The isolated nanobody, conjugate construct of the isolated nanobody, or antigen-binding domain fragment thereof of claim 1 comprising a complementary deoxyribonucleic acid sequence SEQ ID NO:01 or a complementary deoxyribonucleic acid sequence SEQ ID NO:03.

6. The isolated nanobody, conjugate construct of the isolated nanobody, or antigen-binding domain fragment thereof of claim 1 that specifically binds to mesothelin with an affinity of 15 nM or 30 nM.

7. The isolated nanobody, conjugate construct of the isolated nanobody, or antigen-binding domain fragment thereof of claim 1 that is an isolated camelid heavy-chain only antibody that specifically binds to mesothelin.

8. The isolated nanobody, conjugate construct of the isolated nanobody, or antigen-binding domain fragment thereof of claim 1, that is an immune-conjugate, a chimeric antibody, a chimeric antigen receptor T-cell receptor, an antibody-drug conjugate, or an antibody-label conjugate having variable region sequences comprising:
the CDR1 comprising the sequence SEQ ID NO:05, the CDR2 comprising the sequence SEQ ID NO:06 and the CDR3 comprising the sequence SEQ ID NO:07, or
the CDR1 comprising the sequence SEQ ID NO:08, the CDR2 comprising the sequence SEQ ID NO:09 and the CDR3 comprising the sequence SEQ ID NO:10.

9. The isolated nanobody, conjugate construct of the isolated nanobody, or antigen-binding domain fragment thereof of claim 1 as expressed by a cultured cell.

10. The isolated nanobody, conjugate construct of the isolated nanobody, or antigen-binding domain fragment thereof of claim 1 as encoded by an expression vector.

11. An isolated camelid heavy-chain only antibody or conjugate construct thereof that specifically binds to mesothelin, comprising:
a complementary determining region (CDR)1 comprising sequence SEQ ID NO:05 or sequence SEQ ID NO:08;
a CDR2 comprising sequence SEQ ID NO:06 or sequence SEQ ID NO:09; and
a CDR3 comprising sequence SEQ ID NO:07 or sequence SEQ ID NO:10.

12. The isolated camelid heavy-chain only antibody or conjugate construct thereof of claim 11, comprising an amino acid sequence SEQ ID NO:02 or amino acid sequence SEQ ID NO:04.

13. The isolated camelid heavy-chain only antibody or conjugate construct thereof of claim 11, comprising:
the CDR1 comprising the sequence SEQ ID NO:05, the CDR2 comprising the sequence SEQ ID NO:06 and the CDR3 comprising the sequence SEQ ID NO:07; and
an incorporated C-terminal cysteine.

14. The isolated camelid heavy-chain only antibody or conjugate construct thereof of claim 11, comprising:
the CDR1 comprising the sequence SEQ ID NO:05, the CDR2 comprising the sequence SEQ ID NO:06 and the CDR3 comprising the sequence SEQ ID NO:07; and
a biotin covalently attached to the isolated camelid heavy-chain only antibody or conjugate construct thereof.

15. An isolated polynucleotide compound encoding a human anti-mesothelin antigen binding domain, or cells comprising the polynucleotides, the human anti-mesothelin antigen binding domain comprising a complementary deoxyribonucleic acid sequence SEQ ID NO:01 or a complementary deoxyribonucleic acid sequence SEQ ID NO:03.

16. A method comprising:
contacting a biological sample with an isolated nanobody or a conjugate construct of the isolated nanobody that specifically binds to human mesothelin, the isolated nanobody or the conjugate construct of the isolated nanobody comprising:
a complementary determining region (CDR)1 comprising sequence SEQ ID NO:05 or sequence SEQ ID NO:08;
a CDR2 comprising sequence SEQ ID NO:06 or sequence SEQ ID NO:09; and
a CDR3 comprising sequence SEQ ID NO:07 or sequence SEQ ID NO:10; and
detecting binding of the isolated nanobody or the conjugate construct of the isolated nanobody to mesothelin present in the biological sample.

17. The method of claim 16, further including forming the conjugate construct of the isolated nanobody by incorporating a C-terminal cysteine to the nanobody.

18. The method of claim 16, further including forming the conjugate construct of the isolated nanobody by covalently attaching a biotin to the isolated nanobody.

19. The method of claim 16, further including diagnosing a subject with a mesothelin-associated condition in response to an increase in binding of the nanobody or the conjugate construct to the biological sample as compared to the binding of the nanobody or conjugate construct to a control indicative of a non-diseased sample.

20. The method of claim 16, where detecting specific binding of the isolated nanobody or the conjugate construct of the isolated nanobody to human mesothelin present in the biological sample, and the method further including imaging the biological sample in vivo or in vitro.


SEQUENCE LISTING (10 sequences)

SEQ ID NO: 1 - DNA, 292 bp, Lama glama
caccctggggggtctctgagactctcctgtgcagcctctggaatcgacctcagtctttatcgcatgcgctggtatcgccaggctccaggaaaggagcgcgacttggtcgcacttataactgatgatggtacttcgtactatgaagactccgtgaagggccgattcaccatcaccagggacaatccctcgaacaaggtgtttctgcaaatgaacagcctgaaacctgaggacacggccgtctattactgtaatgcagagacgcctttatcgccggtcaactactggggccagg

SEQ ID NO: 2 - protein, 97 aa, Lama glama
HPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQ

SEQ ID NO: 3 - DNA, 293 bp, Lama glama
gcaggctgggggctctctgagactctcctgtgcaccctctggaagcatcttcggtatccgtaccatggactggtaccgccaggctccagggaaggagcgcgagttggtcgcacgaattacgatggatggtcgggtattccatgcagactccgtgaagggccgattctccggctccagagacggcgcctcgaacgcggtgtatctgcaaatgaacagcctgaaacctgacgacacggccgtctattactgtcgatatagtggcttaacctcaagggaggactactggggcccgg

SEQ ID NO: 4 - protein, 97 aa, Lama glama
QAGGSLRLSCAPSGSIFGIRTMDWYRQAPGKERELVARITMDGRVFHADSVKGRFSGSRDGASNAVYLQMNSLKPDDTAVYYCRYSGLTSREDYWGP

SEQ ID NO: 5 - protein, 8 aa, Lama glama
GIDLSLYR

SEQ ID NO: 6 - protein, 7 aa, Lama glama
ITDDGTS

SEQ ID NO: 7 - protein, 11 aa, Lama glama
NAETPLSPVNY

SEQ ID NO: 8 - protein, 8 aa, Lama glama
GSIFGIRT

SEQ ID NO: 9 - protein, 7 aa, Lama glama
ITMDGRV

SEQ ID NO: 10 - protein, 11 aa, lama glama
RYSGLTSREDY